(19) United States
(12) Patent Application Publication
(10) Pub. No.: US 2018/0193334 A1
(43) Pub. Date: Jul. 12, 2018

(54) PHARMACEUTICAL COMPOSITION OF VORTIOXETINE OR SALT THEREOF, AND PREPARATION METHOD THEREFOR

(71) Applicants: Changzhou Fangnan Pharmaceuticals, Ltd., Changzhou (CN); Changzhou Ainuoxinrui Pharmaceuticals, Ltd., Changzhou (CN)

(72) Inventors: Xini Zhang, Green Brook, NJ (US); Zhigang Xiong, Changzhou (CN)

(73) Assignees: Changzhou Fangnan Pharmaceuticals, Ltd., Changzhou (CN); Changzhou Ainuoxinrui Pharmaceuticals, Ltd., Changzhou (CN)

(21) Appl. No.: 15/914,587

(22) Filed: Mar. 7, 2018

Related U.S. Application Data
(63) Continuation of application No. PCT/CN2016/098095, filed on Sep. 5, 2016.

(30) Foreign Application Priority Data
Sep. 7, 2015 (CN) 201510562105.0
Jun. 25, 2016 (CN) 201610474113.4
Jul. 21, 2016 (CN) 201610581662.1

(51) Int. Cl. A61K 31/495

(52) U.S. Cl. CPC A61K 31/495

(57) ABSTRACT

Provided are a pharmaceutical composition of an amorphous vortioxetine or a salt thereof, and a preparation method therefor. The pharmaceutical composition comprises an amorphous vortioxetine or a pharmaceutically acceptable salt thereof and two or more pharmaceutical excipients. The pharmaceutical composition has good stability and dispersity, and enhances the dissolution of vortioxetine or the pharmaceutically acceptable salt thereof, thereby improving the bioavailability of the medicinal formulation and the body absorption of the medicine.

### FIELD OF THE TECHNOLOGY

The present invention belongs to the field of pharmaceutical preparation, in particular to a composition of vortioxetine or its pharmaceutically acceptable salt and a pharmaceutical excipient, a preparation method thereof and the use of the above composition to prepare a medicament for treating affective disorder.

### BACKGROUND TECHNOLOGY

Vortioxetine has the chemical name 1-[2-(2,4-methylphenylthio) phenyl] piperazine hydrobromide under the trade name Brintellix, is a serotonin reuptake inhibitor developed by Lundbeck and Takeda. Vortioxetine hydrobromide was approved by the US Food and Drug Administration on Sep. 30, 2013 for the treatment of major depressive disorder. A report released by Decision Resources, a leading global pharmaceutical and healthcare research and forecasting firm, predicts that vorteoxine will reach the top five markets in the United States, Japan and the EU (France, Germany, Italy, Spain, Britain) by 2022 and will become a blockbuster. Based on the data available to date, vortioxetine is expected to be the most successful new drug in the unipolar depression market, given its positive effect on cognition and tolerable side effects.

However, there are many crystalline forms of vortioxetine or its pharmaceutically acceptable salt. Chinese patent CN 101472906 discloses the crystalline forms of vortioxetine free base and various pharmaceutically acceptable salts, including α-crystalline form, β-crystalline form and γ-crystalline form of vortioxetine hydrobromide. The marketed crystalline form is β-crystalline form of vortioxetine hydrobromide and is also the most thermodynamically stable form of hydrobromide salt.

The solubility of vortioxetine free base in water is very low, only 0.1 mg/ml. The salt of vortioxetine free base with hydrobromic acid can significantly improve its solubility in water, but still only 1.2 mg/ml.

The solid form of the drug directly affects the dissolution rate of the drug substance, the dissolution rate and the bioavailability of the drug substance. In order to improve the bioavailability of the drug, and hence reduce the dosage and reduce the side effects, new solid forms of the drug are usually developed. Therefore, to develop a solid form with higher drug solubility and better bioavailability is necessary.

In addition to the crystalline state of a drug, there are amorphous states. The amorphous state of a drug, as a special form of the solid material, plays an important role in the preparation of drugs. Amorphous drugs can not only be widely used in pharmaceutical preparations, but also by a variety of technical approaches to improve the stability of amorphous drugs, making them the drugs with good quality.

Patent WO2014/177491 reports a composition which adsorbs amorphous vortioxetine hydrobromide onto an inorganic carrier. Vortioxetine hydrobromide in this composition was in an amorphous state with a certain increase in dissolution compared to the crystalline form of vortioxetine hydrobromide. However, the adsorption capacity of the inorganic adsorbent to the active ingredient-vortioxetine hydrobromide is not very high, so a large amount of inorganic adsorbent needs to be added to make vortioxetine hydrobromide into an amorphous state. The patent WO2016/062860 also provides a composition containing amorphous vortioxetine hydrobromide by first adsorbing vortioxetine hydrobromide on the adsorbent, where the other component forms a new combination. In this composition, vortioxetine hydrobromide is in the amorphous state. The technical solution is to make the active ingredient to be in an amorphous state and also add a large amount of adsorbent. Adsorbent on the adsorption capacity of the active ingredient is very strong, and the release of the drug will be impacted greatly. However, the improvement of dissolution is not obvious; in the same time, the inorganic adsorbent needs other organic materials together for granulation and tableting. A large amount of adsorbent will require a larger amount of organic excipients, which will create some difficulties in the formulation of tablets, which in turn affects the bioavailability and efficacy of the drug.

Due to the lack of bioavailability of vortioxetine hydrobromide and the potential applications of amorphous active pharmaceutical ingredients in pharmaceutical preparations, it is very necessary to look for new amorphous vortioxetine hydrobromide and its preparation method.

### DESCRIPTION OF THE INVENTION

The purpose of the present invention is to provide a composition of vortioxetine or its pharmaceutically acceptable salt and a pharmaceutical excipient and a preparation method thereof. The obtained composition provides vortioxetine or its pharmaceutically acceptable salt in amorphous form with good stability and dispersibility. The composition of vortioxetine or its pharmaceutically acceptable salt and a pharmaceutical excipient increases the dissolution of vortioxetine or its pharmaceutically acceptable salt, which is not limited by the drying process nor by the variety of the solvent and the solvent amount. It is easy to operate, low cost, and easy to implement. Industrial scale production can be achieved.

In order to achieve the above goal, the technical solution of the present invention is as follows:

A pharmaceutical composition of vortioxetine or its salt, wherein the composition comprises vortioxetine or its pharmaceutically acceptable salt and two or more kinds of pharmaceutical excipients. The weight ratio of vortioxetine or its pharmaceutically acceptable salt to the total pharmaceutical excipients is from 1:0.1˜400, wherein the vortioxetine or its pharmaceutically acceptable salt in the composition is in an amorphous state. In the X-ray powder diffraction spectrum of the composition, no characteristic peak of vortioxetine or its pharmaceutically acceptable salt crystal is observed after removing the background peak of the pharmaceutical excipient.

Furthermore, the pharmaceutical excipients are selected at least one from the group consisting of excipients, propellants, solubilizers, solubilizers, emulsifiers, colorants, binders, disintegrants, fillers, lubricants, wetting agents, tonicity adjusting agents, stabilizers, glidants, flavoring agents, preservatives, suspending agents, coating materials, flavoring agents, antiblocking agents, integrators, penetration enhancers, pH adjusting agents, buffering agents, plasticizers, surface-active agents, foaming agents, defoamers, thickeners, coating agents, humectants, absorbents, diluents, flocculants and deflocculants, antioxidants, adsorbents, filter aids, release retardants.

Preferably, at least one of the pharmaceutical excipients is selected from the group consisting of hydroxypropylmethylcellulose, hydroxypropylcellulose, povidone, polyethylene glycol, ethylcellulose, liposomes, methacrylic acid copolymerization polyvinyl acetate, carboxymethylethylcellulose, carboxymethylcellulose phthalate, hydroxypropylmethylcellulose phthalate, hydroxypropylmethylcellulose acetate succinate, polyacrylic resins, carbopol, alginates, carrageenan, carboxylactones, gums, polyvinyl alcohol, pregelatinized starch, cross-linked starches, sodium carboxymethyl starch, dextrin, polyethylene oxide, chitosan, collagen, cyclodextrin, lactose, galactose, D-mannitol, sorbitol, xylitol, urea, citric acid, tartaric acid, fumaric acid, maleic acid or succinic acid.

Furthermore, the vortioxetine is in the form of its hydrobromide salt, and the pharmaceutical excipient comprises an organic carrier and an adsorbent. Vortioxetine and the organic carrier form a solid dispersion. The solid dispersion and the adsorbent form a composition, wherein the weight ratio of vortioxetine hydrobromide to the organic vehicle is from 1:0.1˜10 and the weight ratio of vortioxetine hydrobromide to the adsorbent is from 1:0.1˜10. Vortioxetine hydrobromide in the composition is in the amorphous state. In the X-ray powder diffraction spectrum of the composition, no characteristic peaks of vortioxetine hydrobromide crystalline in the composition are observed after removing the background peak of the pharmaceutical excipient.

Further, the pharmaceutical excipient comprises an organic vehicle, an adsorbent and a pharmaceutical excipient, wherein the vortioxetine and the organic vehicle form a solid dispersion, and the solid dispersion forms a composition with the adsorbent and the pharmaceutical excipient. The weight of the vortioxetine hydrobromide is 20˜80% of the total weight of the solid dispersion; the weight of the adsorbent is 0.1˜100% of the weight of the solid dispersion; and the weight of the excipient is 0.1%˜200% of the weight of the solid dispersion. Vortioxetine hydrobromide is in an amorphous state, and in the X-ray powder diffraction spectrum of the composition, no characteristic peaks of vortioxetine hydrobromide crystalline are observed after removing the background peak of the pharmaceutical excipient, the organic carrier, and the adsorbent.

Further, the organic vehicle is selected from pharmaceutically acceptable small molecule organic compounds, polymers or copolymers.

Preferably, the organic vehicle is selected at least one from the group consisting of hydroxypropylmethylcellulose, hydroxypropylcellulose, povidone, polyethylene glycol, ethylcellulose, liposomes, methacrylic acid copolymer, polyvinyl acetate, carboxymethylethylcellulose, carboxymethylcellulose phthalate, hydroxypropylmethylcellulose phthalate, hydroxypropylmethylcellulose acetate succinate, polyacrylic acid resin, carboxyvinyl acetate, carrageenan, carboxylactone, gum, polyvinyl alcohol, pregelatinized starch, cross-linked starch, sodium starch glycolate, dextrin, polyethylene oxide, chitosan, collagen, cyclodextrin, lactose, galactose, D-mannitol, sorbitol, xylitol, urea, citric acid, tartaric acid, fumaric acid, maleic acid and succinic acid.

Also, the adsorbent is selected at least one from silica, alumina, titania, magnesia, calcium carbonate, and zinc oxide.

Further, the pharmaceutical excipient is selected at least one from the group consisting of excipients, propellants, solubilizers, solubilizers, emulsifiers, colorants, binders, disintegrants, fillers, lubricants, wetting agents, tonicity adjusting agents, stabilizers, glidants, flavoring agents, preservatives, suspending agents, coating materials, flavoring agents, antiblocking agents, integrators, penetration enhancers, pH adjusting agents, buffering agents, plasticizers, active agents, foaming agents, defoamers, thickeners, coating agents, humectants, absorbents, diluents, flocculants and deflocculants, antioxidants, adsorbents, filter aids, release retardants.

The preparation method of pharmaceutical composition of vortioxetine or its salt in present invention comprises the following steps:
1) Mixing vortioxetine or its pharmaceutically acceptable salt with a pharmaceutical excipient and heating to melt the pharmaceutical excipient, wherein the weight ratio of vortioxetine or its pharmaceutically acceptable salt to the total pharmaceutical excipients 1:0.1˜100;
2) After being uniformly mixed and cooled, the obtained mixture is pulverized to obtain a composition of vortioxetine in an amorphous state or its pharmaceutically acceptable salt and a pharmaceutical excipient.

The present invention provides a method for preparing another composition of vortioxetine or its pharmaceutically acceptable salt and a pharmaceutical excipient which comprises the following steps:

1) Vortioxetine or its pharmaceutically acceptable salt and a pharmaceutically acceptable excipient are mixed in a solvent at a temperature of −50˜450° C. to form a solution containing vortioxetine or its pharmaceutically acceptable salt and a pharmaceutical excipient, wherein the weight ratio of vortioxetine or its pharmaceutically acceptable salt to solvent is 0.001˜400:1, the ratio of vortioxetine or its pharmaceutically acceptable salt to the pharmaceutical excipient is 1:0.1˜100;

2) Removing the solvent in the solution or suspension obtained in the step 1) to obtain a composition of the vortioxetine in amorphous form or its pharmaceutically acceptable salt and a pharmaceutical excipient.

Further, in the two preparation methods, at least one of the pharmaceutical excipients is selected at least one from the group consisting of excipients, propellants, solubilizers, co-solvents, emulsifiers, colorants, binders, disintegrants, fillers, lubricants, wetting agents, tonicity adjusting agents, stabilizers, glidants, flavoring agents, preservatives, suspending agents, coating materials, flavoring agents, antiblocking agents, integration agents, penetration promoter, pH regulators, buffers, plasticizers, surfactants, foaming agents, defoamers, thickeners, coating agents, humectants, absorbents, diluents, flocculants and deflocculants, antioxidants, adsorbant agents, drainage aids or release retardants.

Preferably, the pharmaceutical excipients is selected at least one from the group consisting of hydroxypropylmethylcellulose, hydroxypropylcellulose, povidone, polyethylene glycol, ethylcellulose, liposomes, methacrylic acid copolymerization, polyvinyl acetate, carboxymethylethylcellulose, carboxymethylcellulose phthalate, hydroxypropylmethylcellulose phthalate, hydroxypropylmethylcellulose acetate succinate, polyacrylic resins, carbopol, alginates, carrageenan, carboxylactones, gums, polyvinyl alcohol, pregelatinized starch, cross-linked starches, sodium carboxymethyl starch, dextrin, polyethylene oxide, chitosan, collagen, cyclodextrin, lactose, galactose, D-mannitol, sorbitol, xylitol, urea, citric acid, tartaric acid, fumaric acid, maleic acid or succinic acid.

In addition, in step 1), the solvent is selected from the group consisting of alcohols having 12 or fewer carbon atoms, phenols, ethers, halogenated hydrocarbons, ketones, aldehydes, nitriles, amides, sulfones, sulfoxides, water; in step 2) the method of removing the solvent comprises: evaporation, vacuum evaporation, spray drying, lyophilization, hot melt extrusion, filtration, centrifugation or agitation film drying.

A preparation method of a combination of amorphous vortioxetine hydrobromide and pharmaceutical excipients comprises the following steps:
1) Mixing the vortioxetine hydrobromide, the organic vehicle and the adsorbent in a solvent at a temperature of −50˜150° C. to form a solution or suspension of the vortioxetine hydrobromide, the organic vehicle and the adsorbent, wherein the weight ratio of vortioxetine hydrobromide to solvent is from 0.001˜100:1, the weight ratio of vortioxetine hydrobromide to organic vehicle is from 1:0.1˜10, and the weight ratio of vortioxetine hydrobromide to adsorbent is 1:0.1˜10;
2) Removing the solvent in the solution or suspension obtained in the step 1) to obtain a pharmaceutical composition of vortioxetine hydrobromide in an amorphous form.

Further, the organic vehicle is selected from pharmaceutically acceptable small molecule organic compounds, polymers or copolymers.

Preferably, the organic vehicle is selected at least one from the group consisting of hydroxypropylmethylcellulose, hydroxypropylcellulose, povidone, polyethylene glycol, ethylcellulose, liposomes, methacrylic acid copolymers, polyvinyl acetate, Carboxymethylethylcellulose, carboxymethylcellulose phthalate, hydroxypropylmethylcellulose phthalate, hydroxypropylmethylcellulose acetate succinate, polyacrylic resin, poly carboxyvinyl, alginate, carrageenan, carboxylactone, gum, polyvinyl alcohol, pregelatinized starch, cross-linked starch, sodium carboxymethyl starch, dextrin, polyethylene oxide, chitosan, butyrate, collagen, cyclodextrin, lactose, galactose, D-mannitol, sorbitol, xylitol, urea, citric acid, tartaric acid, fumaric acid, maleic acid and succinic acid.

Also, the adsorbent is selected from at least one of silica, alumina, titaniam, magnesia, calcium carbonate, and zinc oxide.

In Step 1), the solvent is selected from alcohols having 12 or less carbon atoms, phenols, ethers, halogenated hydrocarbons, ketones, aldehydes, nitriles, amides, sulfones, sulfoxides, carboxylic acids and water; in step 2) the method of removing the solvent comprises: evaporation, vacuum evaporation, spray drying, lyophilization, hot melt extrusion, filtration, centrifugation or stirring film drying.

A preparation method of a pharmaceutical composition of amorphous vortioxetine hydrobromide comprises the following steps:
1) Mixing the vortioxetine hydrobromide, at least one organic carrier, at least one adsorbent and at least one pharmaceutically acceptable excipient in a solvent at a mixing temperature of −50˜150° C. to form a solution containing vortioxetine hydrobromide, an organic carrier, a solution or suspension of an adsorbent and a pharmaceutically acceptable excipient, wherein the weight ratio of vortioxetine hydrobromide to solvent is from 0.001˜100:1, and the weight of vortioxetine hydrobromide salt is 20%˜80% of the total weight of the solid dispersion, and the weight of the pharmaceutical excipient is 0.1%˜80% of the weight of the solid dispersion;
2) Removing the solvent in the solution or suspension obtained in the step 1) to obtain a pharmaceutical composition of vortioxetine hydrobromide in an amorphous form.

The present invention also provides another method for preparing a pharmaceutical composition containing amorphous vortioxetine hydrobromide, comprising the following steps:
1) Mixing the vortioxetine hydrobromide, at least one organic carrier, at least one adsorbent, and at least one pharmaceutically acceptable excipient and a solvent in a fluidized bed at a mixing temperature of from 0˜150° C. to form a solution or suspension of vortioxetine hydrobromide, an organic vehicle, an adsorbent and a pharmaceutically acceptable excipient, wherein the weight ratio of vortioxetine hydrobromide to solvent is 0.001˜400:1. The weight of vortioxetine hydrobromide is 20%˜80% of the total weight of the solid dispersion, the weight of the pharmaceutical excipient is 0.1%˜80% of the weight of the solid dispersion;
2) Removing the solvent in the mixture obtained in step 1) to obtain the formulation of vortioxetine hydrobromide in amorphous form.

Further, the organic vehicle of the pharmaceutical excipient is selected from the group consisting of a pharmaceutically acceptable small molecule organics, polymers or copolymers.

Preferably, the organic vehicle is selected at least one from the group consisting of hydroxypropylmethylcellulose, hydroxypropylcellulose, povidone, polyethylene glycol, ethylcellulose, liposomes, methacrylic acid copolymer, polyvinyl acetate, Carboxymethylethylcellulose, carboxymethylcellulose phthalate, hydroxypropylmethylcellulose phthalate, hydroxypropylmethylcellulose acetate succinate, polyacrylic acid resin, carboxyvinyl acetate, carrageenan, carboxylactone, gum, polyvinyl alcohol, pregelatinized starch, crosslinked starch, sodium starch glycolate, dextrin, polyethylene oxide, chitosan, chitosan and collagen.

In addition, in step 1), the solvent is selected from the group consisting of alcohols having 12 or less carbon atoms, phenols, ethers, halogenated hydrocarbons, ketones, aldehydes, nitriles, amides, sulfones, sulfoxides, water; in step 2) the method of removing the solvent comprises: evaporation, vacuum evaporation, spray drying, lyophilization, hot melt extrusion, filtration, centrifugation or agitation film drying.

The present invention also provides the use of a pharmaceutical composition containing amorphous vortioxetine hydrobromide for the treatment of mental disorders including mood disorders, depression, anxiety disorders, post-traumatic stress disorder, depression with cognitive impairment, Alzheimer's disease, depression with residual symptoms, habitual pain and eating disorders.

The composition in the present invention means a mixture, a complex, a copolymer, a coprecipitate, a eutectic, a solid dispersion, a solvate and a hydrate.

In the present invention, the pharmaceutical excipients are excipients and additives used in the manufacture of medicines and prescriptions, including excipients, propellants, solubilizers, solubilizers, emulsifiers, colorants, binders, disintegrants, fillers, lubricants, wetting agents, tonicity adjusting agents, stabilizers, glidants, flavoring agents, preservatives, suspending agents, coating materials, flavoring agents, antiblocking agents, integration agents, penetration promoter, pH adjusting agents, buffering agents, plasticizers, surfactants, foaming agents, defoamers, thickeners, inclusion agents, humectants, absorbents, diluents, flocculants and deflocculants, antioxidants, adsorbents, filter aids, release retardants and so on.

The composition of the vortioxetine or its pharmaceutically acceptable salt and the pharmaceutical excipient in the invention is obtained by removing the background peak of the pharmaceutical adjuvant from the X-ray powder diffraction spectrum expressed in degrees 2θ using Cu-Kα radiation. The characteristic peaks of crystalline form of vortioxetine or its pharmaceutically acceptable salt are not observed, indicating that vortioxetine or its pharmaceutically acceptable salt is in an amorphous state. In the prior art, the crystalline state of vortioxetine or its pharmaceutically acceptable salt is generally used, and no report of its amorphous state has been reported. Generally, due to the orderly and periodic arrangement of crystalline substance molecules, the energy of the intermolecular interaction is reduced and the energy is lower. However, the vortioxetine or its pharmaceutically acceptable salt in the present invention is in an amorphous state. The molecule is in a highly disordered state, the surface free energy of the substance is larger, the molecules in the solid material have higher energy than the molecules in the crystalline solid material, the dispersion is easier, the dissolution rate is increased, and bioavailability of vortioxetine or its pharmaceutically acceptable salt is improved.

In the invention, after the vortioxetine or its pharmaceutically acceptable salt and the excipient are uniformly mixed, the drug molecules are blocked by the “solid dispersant” method and the drug molecules are blocked through the polymer network structure of the pharmaceutical excipients, and the crystalline occurs, keeping it dispersed and amorphous. The pharmaceutical excipients in the present invention use two or more kinds of pharmaceutical excipients. Compared with a single excipient, a variety of pharmaceutical excipients mutual compatibility, can disperse better, block drug molecules and inhibit the role of crystallization. For example, in view of this structural feature of vortioxetine or its pharmaceutically acceptable salt having a plurality of amine groups, the present invention can introduce polyhydroxy alcohols into pharmaceutical excipient, drug molecules and hydroxyl groups of alcohols are easy to form hydrogen bonds, resulting in a strong interaction, can enhance the drug dispersion in the excipients, and can inhibit the crystallization of drug molecules better. In addition, a variety of pharmaceutical excipients can also play different roles in pharmaceutical formulations, which facilitates the development of pharmaceutical formulations.

The present invention uses a wide range of applications, low price, good solubility of pharmaceutical excipients, these pharmaceutical excipients and vortioxetine or its pharmaceutically acceptable salts are mixed, not subject to the drying process is not limited by the type of solvent and solvent amount. The combination of evaporation, spray-drying, lyophilization and hot-melt extrusion techniques results in an amorphous form of vortioxetine or its pharmaceutically acceptable salt which increases the pharmacological effects of vortioxetine or its salt. The stability of the amorphous state of the composition, easy to operate, low cost, easy to implement, and industrial production can be achieved.

Compared with the prior art, the beneficial effects of the present invention are as follows:
1) The formulation of the amorphous vortioxetine or its pharmaceutically acceptable salt prepared by the invention and the composition of two or more kinds of pharmaceutical excipients has high dispersibility and stability, and various kinds of pharmaceutical excipients can be used in drug formulations and play a different role in favor of formulation development. After the preparation of the solid preparation and the disintegration, the degree of dispersion of the drug particles can be improved, and the dispersion and dissolution can be increased, which is favorable for the absorption of the drug. Therefore, the amorphous state of the drug dissolution significantly increased, more conducive to the body's absorption of drugs to improve the bioavailability of drugs, so that drugs can play a better therapeutic role in clinical treatment.
2) The preparation method of the composition of the vortioxetine or its pharmaceutically acceptable salt and the pharmaceutical excipient in the amorphous state in the present invention is not limited by the drying process, is not limited by the type of the solvent and the amount of the solvent, and is easy to operate, low cost, easy to implement and industrialized production.
3) The composition of the invention is prepared in the amorphous state of vortioxetine or its pharmaceutically acceptable salt and the pharmaceutical excipient under the conditions of high temperature and high humidity, the substance has no significant change, and no vortioxetine or its pharmaceutically acceptable salts are crystallized out; under accelerated conditions (40±2° C., 75%±5% humidity), the substance has no significant change, no vortioxetine or its pharmaceutically acceptable salts are crystallized out. The amorphous state of the composition of the invention and its pharmaceutically acceptable salts and the pharmaceutical excipient can maintain good physical stability and chemical stability and will have broad application prospects.

### DESCRIPTION OF THE DRAWINGS

FIG. 1 is an X-ray powder diffraction pattern of the composition of amorphous vortioxetine hydrobromide and D-mannitol and Povidone K30 of Example 1 in the present invention.

FIG. 2 is an X-ray powder diffraction pattern of a composition of amorphous vortioxetine hydrobromide and sorbitol and polyacrylic acid resin L100 of Example 12 in the present invention.

FIG. 3 is an X-ray powder diffraction pattern of a composition of amorphous vortioxetine hydrobromide, povidone K30 and colloidal silica (Aerosil 200) of Example 38 in the present invention.

FIG. 4 is an X-ray powder diffraction pattern of a composition of amorphous vortioxetine hydrobromide, hydroxypropylmethylcellulose (HPMC E3) and colloidal silica (Aerosil 200) of Example 39 in the present invention.

FIG. 5 is an X-ray powder diffraction pattern of a composition of the composition of amorphous vortioxetine hydrobromide, povidone K30, microcrystalline cellulose and colloidal silica Aerosil 200 of Example 57 in the present invention.

FIG. 6 is an X-ray powder diffraction pattern of microcrystalline cellulose used in Example 57 in the present invention.

FIG. 7 is an X-ray powder diffraction pattern of the composition of amorphous vortioxetine, hydroxypropylmethylcellulose HPMC E3, mannitol, and colloidal silica Aerosil 200 of Example 58 in the present invention.

FIG. 8 is an X-ray powder diffraction pattern of the composition of the composition of amorphous vortioxetine hydrobromide, Povidone K30, microcrystalline cellulose, and colloidal silica Aerosil 200 of Example 73 in the present invention.

### DETAILED DESCRIPTION

The present invention will be further described with reference to specific embodiments, but the protection scope of the present invention is not limited by the following embodiments.

The X-ray powder diffraction pattern according to the invention was taken on an Ultima IV X-ray diffractometer. The method parameters of the X-ray powder diffraction according to the present invention are as follows:

X-ray powder parameters: Cu-Kα

Kα (Å): 1.5418;

Voltage: 40 kV;

Current: 40 mA;

Divergence slit: automatic;

Scanning mode: continuous;

Scanning range: from 2.0 to 60.0°;

Sampling period: 0.0200°;

Scanning rate: 60°/min.

Any solid form of vortioxetine hydrobromide can be used in the preparation of the pharmaceutical compositions of the present invention.

The loading of vortioxetine hydrobromide in the pharmaceutical composition was calculated as follows:

Loading rate=The content of vortioxetine hydrobromide in the pharmaceutical composition/the total weight of the pharmaceutical composition.

### Example 1

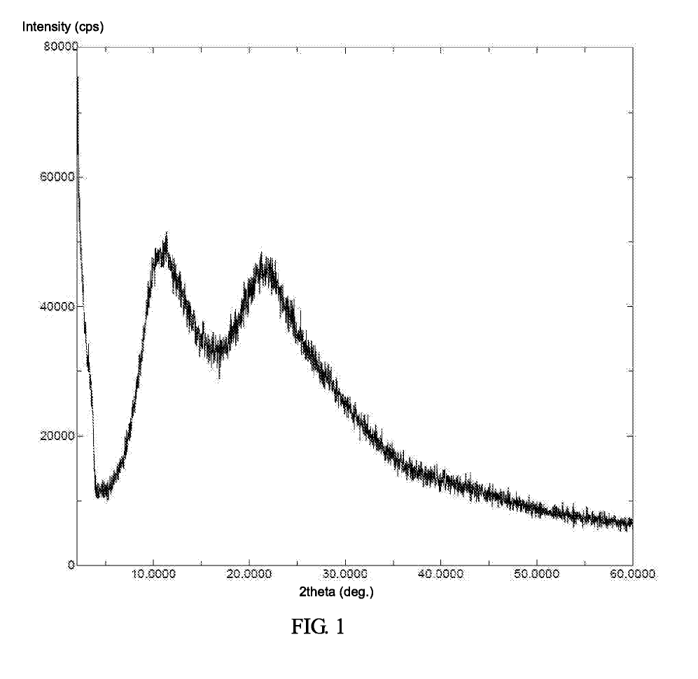

Vortioxetine hydrobromide (50 mg), D-mannitol (50 mg) and povidone K30 (50 mg) were dissolved in methanol (800 μL) and heated to 60° C. to dissolve. The solution was rapidly cooled to −10° C. to precipitate a white solid, which was filtered and dried to obtain a composition of amorphous vortioxetine hydrobromide and D-mannitol and povidone K30 having X-ray Powder diffraction pattern shown in FIG. 1. As can be seen from FIG. 1, the X-ray powder diffraction pattern of this composition, after subtracting the peak of the excipient, did not have the characteristic peak of the vortioxetine hydrobromide crystalline form.

### Example 2

Vortioxetine (50 mg), polyacrylic resin Eudragit L100 (50 mg) and polyethylene glycol 4000 (200 mg) were dissolved in ethanol (600 μl) and water, stirred well and mixed well. The above solution was slowly concentrated to dryness on a rotary evaporator to give a white solid, giving a composition of amorphous vortioxetine and polyacrylic resin Eudragit L100 and polyethylene glycol 4000. The X-ray powder diffraction pattern of the composition, after subtracting the background of the pharmaceutical excipients peak, showed no vortioxetine crystalline characteristic peaks.

### Example 3

Vortioxetine hydrochloride (2 g), lactose (2 g) and polyethylene glycol 8000 (10 g) were added to water (300 ml) and heated to 60° C. to dissolve. The above solution was dried with a JISL mini spray dryer LSD-48 to maintain an inlet temperature of 60° C. and an outlet temperature of 50° C. The outlet material was collected to give a white solid which was further dried in vacuo to afford amorphous vortioxetine hydrochloride and lactose and poly Ethylene glycol 8000 composition. The X-ray powder diffraction pattern of this composition, after subtracting the peak of the excipient, did not have the characteristic peak of the vortioxetine hydrochloride crystalline form.

### Example 4

Vortioxetine, its hydrobromide salt (1 g), galactose (1 g) and hydroxypropylmethylcellulose E50 (0.2 g) were added to water (10 ml) and heated to 40° C. to dissolve. The solution was lyophilized to give a white solid, ie, a composition of amorphous vortioxetine hydrobromide and galactose and hydroxypropylmethylcellulose E50. The X-ray powder diffraction pattern of this composition, after subtracting the peak of the excipient, did not have the characteristic peak of the vortioxetine hydrobromide crystalline form.

### Example 5

Vortioxetine (5 g), urea (10 g) and polyethylene glycol 8000 (50 g) were heated to melt and rapidly cooled to room temperature with stirring to give a white solid. The above solid was pulverized to obtain a white powdery solid, ie, a composition of amorphous vortioxetine and urea and polyethylene glycol 8000. The X-ray powder diffraction pattern of this composition, after subtracting the peak of the excipient, did not have the characteristic peak of the vortioxetine crystalline form.

### Example 6

Vortioxetine (1 g), ethanol (0.1 g), sorbitol (1 g) and polyethylene glycol 10000 (20 g) were heated to 240° C., mixed well and rapidly cooled to room temperature to give a white solid. The above solid was pulverized to obtain a white powdery solid, ie, a composition of amorphous vortioxetine and sorbitol and polyethylene glycol 10000. The X-ray powder diffraction pattern of this composition, after subtracting the peak of the excipient, did not have the characteristic peak of the vortioxetine crystalline form.

### Example 7

The mixture of vortioxetine fumarate (1 g), fumaric acid (2 g), tetrahydrofuran (10 g), ethanol (20 g) and liposomes (4 g) was heated to 60° C. with stirring, mixed well, evaporated the solvent in vacuo and cool to room temperature to give a white solid, ie, the composition of amorphous vortioxetine fumarate with fumaric acid and liposomes. The X-ray powder diffraction pattern of this composition, after subtracting the peak of the excipient, did not have the characteristic peak of the vortioxetine fumarate crystalline form.

### Example 8

The mixture of vortioxetine hydrobromide (1 g), methanol (20 g), polyacrylic acid resin Eudragit L100 (2 g) and methacrylic acid copolymer type A (4 g) was heated to 50° C. and stirred, dissolved, the solvent was removed in vacuo and the solution was cooled to room temperature to give a white solid, ie, a combination of amorphous vortioxetine hydrobromide and polyacrylic resin Eudragit L100 and methacrylic copolymer Type A. The X-ray powder diffraction pattern of this composition, after subtracting the peak of the excipient, did not have the characteristic peak of the vortioxetine hydrobromide crystalline form.

### Example 9

The mixture of vortioxetine (1 g), methanol (20 g), pregelatinized starch (1 g) and ethylcellulose (2 g) was heated to 30° C., stirred and mixed well and the solvent was evaporated in vacuo. Cooling to room temperature gave a white solid, ie, a combination of amorphous vortioxetine with pregelatinized and ethylcellulose. The X-ray powder diffraction pattern of this composition, after subtracting the peak of the excipient, did not have the characteristic peak of the vortioxetine crystalline form.

### Example 10

A mixture of vortioxetine hydrobromide (1 g), methanol (20 g), xylitol (2 g) and hydroxypropylcellulose SSL (4 g) was heated to 30° C., stirred and dissolved, vacuum evaporation of the solvent by evaporation and cooling to room temperature gave a white solid, ie, a combination of amorphous vortioxetine hydrobromide and xylitol and hydroxypropylcellulose SSL. The X-ray powder diffraction pattern of this composition, after subtracting the peak of the excipient, did not have the characteristic peak of the vortioxetine hydrobromide crystalline form.

### Example 11

The mixture of vortioxetine hydrobromide (1 g), methanol (20 g), water (10 g), citric acid (1 g) and polyvinyl acetate (4 g) was heated to 30° C. with stirring to dissolve, the solvent was evaporated in vacuo and cooled to room temperature to give a white solid, ie, a combination of amorphous vortioxetine hydrobromic acid and citric acid and polyvinyl acetate. The X-ray powder diffraction pattern of this composition, after subtracting the peak of the excipient, did not have the characteristic peak of the vortioxetine hydrobromide crystalline form.

### Example 12

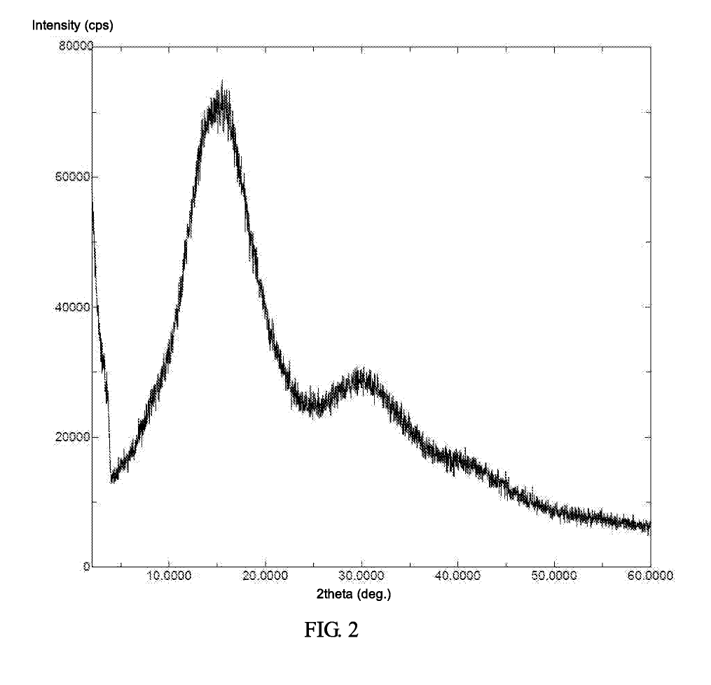

Vortioxetine hydrobromide (50 mg), sorbitol (100 mg) and polyacrylic resin Eudragit L100 (100 mg) were added to methanol (750 μl) and the mixture was stirred and dissolved at room temperature. The above solution was slowly concentrated to dryness in a rotary evaporator to give a white solid, ie, a combination of amorphous vortioxetine hydrobromide and sorbitol and polyacrylic acid resin Eudragit L100 having X-ray powder diffraction as FIG. 2 shows. It can be seen from FIG. 2, the X-ray powder diffraction pattern of this composition, after subtracting the peak of the excipient, did not have the characteristic peak of the vortioxetine hydrobromide crystalline form.

### Example 13

Vortioxetine (50 mg), carboxymethylcellulose phthalate Agucoat CPD (2 mg), and polyacrylic acid resin Eudragit S100 (3 mg) were added to methanol (4 mL) and ethyl acetate), at −30° C. stirring to dossolve. The solution was slowly concentrated to dryness in a rotary evaporator to give a white solid, which was precipitated as a white solid with stirring, ie, amorphous composition of vortioxetine and carboxymethylcellulose phthalate Agucoat CPD and polyacrylic resin Eudragit S100. The X-ray powder diffraction pattern of this composition, after subtracting the peak of the excipient, did not have the characteristic peak of the vortioxetine crystalline form.

### Example 14

Vortioxetine (50 mg), dextrin (50 mg) and carbopol 940 (50 mg) were added to methanol (4 ml) and tetrahydrofuran (1 ml), and the mixture was stirred and homogenized at −30° C. The above solution was slowly concentrated to dryness in a rotary evaporator to give a white solid, which was precipitated as a white solid with stirring, ie, the combination of amorphous vortioxetine and dextrin and Carbomer 940. The X-ray powder diffraction pattern of this composition, after subtracting the peak of the excipient, did not have the characteristic peak of the vortioxetine or its pharmaceutically acceptable salt crystalline form.

### Example 15

Vortioxetine hydrobromide (50 mg), β-cyclodextrin (100 mg) and pregelatinized starch Pharma-Gel (100 mg) were added to methanol (4 ml) and water (1 ml). Mixed well at room temperature. The solution was slowly concentrated to dryness in a rotary evaporator to give a white solid, which was precipitated as a white solid with stirring, ie, a combination of amorphous vortioxetine hydrobromide and β-cyclodextrin and pre-gelatinized starch Pharma-Gel. In the X-ray powder diffraction pattern of the composition, no characteristic peak of the vortioxetine hydrobromide crystalline form was observed after subtracting the background peak of the pharmaceutical excipient.

### Example 16

Vortioxetine (50 mg), β-cyclodextrin (100 mg) and highly branched cross-linked starch (50 mg) were added to methanol (4 ml) and water (1 ml), stirred at room temperature for dissolution. The solution was slowly concentrated to dryness on a rotary evaporator to give a white solid, which was precipitated as a white solid with stirring, ie, a combination of amorphous vortioxetine and β-cyclodextrin (100 mg) and high-branched cross-linked starch. The X-ray powder diffraction pattern of this composition, after subtracting the peak of the excipient, did not have the characteristic peak of the vortioxetine crystalline form.

### Example 17

Vortioxetine nitrate (50 mg), maleic acid (100 mg) and sodium carboxymethyl cellulose SCMC (500 mg) were added to dimethylsulfoxide (5 ml) and the mixture was stirred at room temperature for dissolution. The above solution was slowly concentrated to dryness to give a white solid, ie, a composition of amorphous vortioxetine nitrate and maleic acid and sodium carboxymethylcellulose SCMC. The X-ray powder diffraction pattern of this composition, after subtracting the peak of the excipient, did not have the characteristic peak of the vortioxetine nitrate crystalline form.

### Example 18

Vortioxetine (50 mg), polyethylene glycol 4000 (100 mg) and chitosan (400 mg) were added to ethanol (5 ml) and the mixture was stirred and dissolved at room temperature. The above solution was stirred in a rotary evaporator slowly concentrated to dryness to give a white solid, ie, a combination of amorphous vortioxetine and polyethylene glycol 4000 and chitosan. The X-ray powder diffraction pattern of this composition, after subtracting the peak of the excipient, did not have the characteristic peak of the vortioxetine crystalline form.

### Example 19

Vortioxetine hydrobromide (50 mg), D-mannitol (50 mg) and sodium starch glycolate Explotab (500 mg) were added to ethanol (5 ml), stirred and mixed at room temperature, and the above solution slowly concentrated to dryness in a rotary evaporator to give a white solid, ie, a combination of amorphous vortioxetine hydrobromide with D-mannitol and sodium starch glycolate Explotab. The X-ray powder diffraction pattern of this composition, after subtracting the peak of the excipient, did not have the characteristic peak of the vortioxetine hydrobromide crystalline form.

### Example 20

Vortioxetine hydrochloride (50 mg), povidone K90 (100 mg) and alginate E401 (100 mg) were added to ethanol (5 ml), and stirred and mixed at room temperature. The above solution was slowly concentrated to dryness in a rotary evaporator to give a white solid, ie, a combination of amorphous vortioxetine hydrochloride, povidone K90 and alginate E401. The X-ray powder diffraction pattern of this composition, after subtracting the peak of the excipient, did not have the characteristic peak of the vortioxetine hydrochloride crystalline form.

### Example 21

Vortioxetine hydrobromide (50 mg), L-tartaric acid (100 mg) and carboxymethylcellulose phthalate Agucoat CPD (1 g) were suspended in methanol (30 ml) and heated to 50° C. with stir mixing. The above solution was slowly concentrated in a rotary evaporator to remove most of the solvent, filtered and dried to give a white solid, ie, amorphous vortioxetine hydrobromide with L-tartaric acid and carboxymethylcellulose phthalate Ester Agucoat CPD composition. The X-ray powder diffraction pattern of this composition, after subtracting the peak of the excipient, did not have the characteristic peak of the vortioxetine hydrobromide crystalline form.

### Example 22

Vortioxetine (50 mg), gum Galactosol (100 mg) and carrageenan E407 (100 mg) were suspended in methanol (30 ml), heated to 50° C., stirred and mixed well, and the solution was slowly concentrated in a rotary evaporator to remove most of the solvent, filtered, and dried to give a white solid, ie, a combination of amorphous vortioxetine and Gum Galactosol and Carrageenan E407. The X-ray powder diffraction pattern of this composition, after subtracting the peak of the excipient, did not have the characteristic peak of the vortioxetine hydrobromide crystalline form.

### Example 23

Vortioxetine hydrobromide (50 mg), galactose (100 mg) and chitosan (200 mg) were suspended in methanol (50 ml) and heated to 50° C. and mixed well. The above solution was slowly concentrated in a rotary evaporator to remove most of the solvent, filtered and dried to provide a white solid, ie, a combination of amorphous vortioxetine hydrobromide and galactose and chitosan. The X-ray powder diffraction pattern of this composition, after subtracting the peak of the excipient, did not have the characteristic peak of the vortioxetine hydrobromide crystalline form.

### Example 24

Vortioxetine (300 mg), liposomes (300 mg) and polyacrylic acid Eudragit E100 (300 mg) were dissolved in ethanol (600 μl), tetrahydrofuran (900 μl) and N, N-dimethyl formamide (600 μl), heated to 50° C. and stirred to dissolve. The solution was cooled down to −30° C., a white solid was precipitated, filtered and dried to obtain amorphous vortioxetine with liposomes and polyacrylic resin Eudragit E100. The X-ray powder diffraction pattern of the composition had no characteristic peak of the vortioxetine crystalline form after subtracting the background peak of the pharmaceutical excipient.

### Example 25

Vortioxetine (30 mg), xylitol (30 mg) and collagen Peptan (200 mg) were dissolved in ethanol (600 μl) and acetonitrile (600 μl) and heated to 50° C. to dissolve. The solution was slowly concentrated in a rotary evaporator to remove most of the solvent, a white solid was precipitated, filtered and dried to give a composition of amorphous vortioxetine with xylitol and collagen Peptan. The X-ray powder diffraction pattern of the composition had no characteristic peak of the vortioxetine crystalline form after subtracting the background peak of the pharmaceutical excipient.

### Example 26

Vortioxetine (30 mg), D-mannitol (30 mg) and gum galactosol (150 mg) were dissolved in methanol (900 μl) and heated to 50° C. to dissolve. The solution was slowly concentrated in a rotary evaporator to remove most of the solvent, a white solid was precipitated, filtered and dried to give a composition of amorphous vortioxetine with D-mannitol and gum galactosol. The X-ray powder diffraction pattern of the composition had no characteristic peak of the vortioxetine crystalline form after subtracting the background peak of the pharmaceutical excipient.

### Example 27

Vortioxetine (30 mg), chitosan (30 mg) and hydroxypropylmethylcellulose phthalate HPMCP (30 mg) were added to ethanol (750 μl) and water (750 μl), heated to 80° C. while stir mixing. The above solution was slowly concentrated in a rotary evaporator to remove the solvent to give a white solid, ie, a composition of amorphous vortioxetine and chitosan and hydroxypropylmethylcellulose phthalate HPMCP. The X-ray powder diffraction pattern of the composition had no characteristic peak of the vortioxetine crystalline form after subtracting the background peak of the pharmaceutical excipient.

### Example 28

Vortioxetine hydrobromide (30 mg), D-mannitol (30 mg) and carboxylactone (300 mg) were added to ethanol (750 μl) and water (750 μl), heated to 80° C. while stir mixing. The above solution was slowly concentrated in a rotary evaporator to remove the solvent to give a white solid, ie, a combination of amorphous vortioxetine hydrobromide with D-mannitol and carboxypelaride. The X-ray powder diffraction pattern of the composition had no characteristic peak of the vortioxetine hydrobromide crystalline form after subtracting the background peak of the pharmaceutical excipient.

### Example 29

Vortioxetine (30 mg), β-cyclodextrin (60 mg) and dextrin Maltrin M100 (60 mg) were added to ethanol (750 μl) and water (750 μl), heated to 80° C. while stir mixing. The solution was slowly concentrated in a rotary evaporator to remove the solvent to give a white solid, ie, a composition of amorphous vortioxetine with β-cyclodextrin and dextrin Maltrin M100. The X-ray powder diffraction pattern of the composition had no characteristic peak of the vortioxetine crystalline form after subtracting the background peak of the pharmaceutical excipient.

### Example 30

Vortioxetine hydrobromide (30 mg), succinic acid (3 mg) and sodium carboxymethylcellulose SCMS (3 mg) were added to water (30 ml) and heated to 100° C. and mixed well. The solution was slowly concentrated in a rotary evaporator to remove the solvent to give a white solid, ie, a combination of amorphous vortioxetine hydrobromide and succinic acid and sodium carboxymethylcellulose SCMC. The X-ray powder diffraction pattern of the composition had no characteristic peak of the vortioxetine hydrobromide crystalline form after subtracting the background peak of the pharmaceutical excipient.

### Example 31

Vortioxetine (5 mg), sorbitol (5 mg) and polyethylene oxide Polyox WSR301 (30 mg) were added to methanol (300 μl) and water (60 μl), heated to 60° C. with stirring and mixed well. The solution was slowly concentrated in a rotary evaporator to remove the solvent to give a white solid, ie, a composition of amorphous vortioxetine with sorbitol and polyethylene oxide Polyox WSR301. The X-ray powder diffraction pattern of the composition had no characteristic peak of the vortioxetine crystalline form after subtracting the background peak of the pharmaceutical excipient.

### Example 32

Vortioxetine hydrobromide (30 mg), sorbitol (20 mg), polyethylene glycol 8000 (20 mg) and polyvinyl alcohol EG-40 (20 mg) were added to methanol (300 μl) and water (60 μl) and the solution was stirred to dissolve at 60° C. The solution was slowly concentrated to remove the solvent in a rotary evaporator to give a white solid, ie, amorphous vortioxetine hydrobromide and sorbitol, polyethylene glycol 8000 and polyvinyl alcohol EG-40. The X-ray powder diffraction pattern of the composition had no characteristic peak of the vortioxetine hydrobromide crystalline form after subtracting the background peak of the pharmaceutical excipient.

### Example 33

Vortioxetine (50 mg), xylitol (50 mg) and hydroxypropylmethylcellulose acetate succinate Agoat MG (1 g) were added to ethanol (10 ml) and water (2 ml). The mixture was stirred and mixed well at 80° C. The above solution was slowly concentrated to dryness in a rotary evaporator to obtain a white solid, that is, a combination of amorphous vortioxetine and Xylitol and hydroxypropylmethylcellulose acetate succinate Agoat MG. The X-ray powder diffraction pattern of the composition showed no characteristic peak of the vortioxetine crystalline form after subtracting the background peak of the pharmaceutical excipient.

### Example 34

Vortioxetine (50 mg), sorbitol (100 mg) and carboxymethylethylcellulose (1 g) were added to ethanol (10 ml) and water (1 ml). The mixture was stirred and mixed well at 80° C. The solution was slowly concentrated to dryness on a rotary evaporator to give a white solid, ie, a combination of amorphous vortioxetine with sorbitol and carboxymethylethylcellulose. The X-ray powder diffraction pattern of the composition showed no characteristic peak of the vortioxetine crystalline form after subtracting the background peak of the pharmaceutical excipient.

### Example 35: Influencing Factors of Compositions of Amorphous Vortioxetine Hydrobromide and D-Mannitol and Povidone K30 Test

Materials: The combination of amorphous vortioxetine hydrobromide obtained in Example 1 with D-mannitol and Povidone K30.

TABLE 1

| | | Condition | | | |
|---|---|---|---|---|---|
| | | Temperature | Temperature | | |
| | | 40° C. ± | 60° C. ± | Humidity | Humidity |
| Test items | Time | 2° C. | 2° C. | 75% ± 5% | 90% ± 5% |
| Related | 0 d | 0.09 | | | |
| substances | 5 d | 0.10 | 0.11 | 0.09 | 0.10 |
| (total | 10 d | 0.12 | 0.14 | 0.10 | 0.11 |
| impurity %) | | | | | |

| | | |
|---|---|---|
| Form (X- | 0 d | After removing the background peaks of the |
| powder | | pharmaceutical excipients, there was no |
| diffraction | | characteristic peak of the vortioxetine |
| pattern) | | hydrobromide crystalline form |
| | 5 d | After removing the background peaks of the |
| | | pharmaceutical excipients, there was no |
| | | characteristic peak of the vortioxetine |
| | | hydrobromide crystalline form |
| | 10 d | After removing the background peaks of the |
| | | pharmaceutical excipients, there was no |
| | | characteristic peak of the vortioxetine |
| | | hydrobromide crystalline form |

Table 1 shows that the amorphous vortioxetine hydrobromide and D-mannitol and Povidone K30 compositions under high temperature and high humidity conditions for 10 days, had no significant changes in the relevant substances, no vortioxetine or its pharmaceutically acceptable salt crystallized out.

### Example 36: Accelerated Testing of Amorphous Vortioxetine Hydrobromide with D-Mannitol and Povidone K30 Compositions

Materials: The combination of amorphous vortioxetine hydrobromide obtained in Example 1 with D-mannitol and Povidone K30.

Experimental conditions: temperature 40° C.±2° C., humidity 75%±5%, the results see table.

TABLE 2

| | | Condition |
|---|---|---|
| Test items | time | Temperature 40° C. ± 2° C., Humidity 75% ± 5% |
| Related | 0 M | 0.09 |
| substances | 1 M | 0.10 |
| (total | 2 M | 0.11 |
| impurity %) | 3 M | 0.12 |
| | 6 M | 0.14 |
| Form (X- | 0 M | After removing the background peaks of the |
| powder | | pharmaceutical excipients, there was no |
| diffraction | | characteristic peak of the vortioxetine |
| pattern) | | hydrobromide crystalline form |
| | 1 M | After removing the background peaks of the |
| | | pharmaceutical excipients, there was no |
| | | characteristic peak of the vortioxetine |
| | | hydrobromide crystalline form |
| | 2 M | After removing the background peaks of the |
| | | pharmaceutical excipients, there was no |
| | | characteristic peak of the vortioxetine |
| | | hydrobromide crystalline form |
| | 3 M | After removing the background peaks of the |
| | | pharmaceutical excipients, there was no |
| | | characteristic peak of the vortioxetine |
| | | hydrobromide crystalline form |
| | 6 M | After removing the background peaks of the |
| | | pharmaceutical excipients, there was no |
| | | characteristic peak of the vortioxetine hydrobromide |
| | | crystalline form |

Table 2 shows that the amorphous vortioxetine hydrobromide of the present invention and D-mannitol and Povidone K30 compositions under accelerated test conditions for 6 months had no significant change in related substances, no vortioxetine hydrobromide crystallized out.

The combination of the amorphous vortioxetine or the pharmaceutically acceptable salt thereof and the pharmaceutical excipient of the invention obviously increases the dissolution rate and is more favorable to improve the bioavailability of the medicine and enable the medicine to better exert the clinical disease treatment. The amorphous composition under the accelerated test conditions (40±2° C., humidity 75%±5%) maintained good physical stability and chemical stability.

### Example 37: Apparent Solubility was Determined

The apparent solubility of the mixture of the composition of the invention and the vortioxetine crystalline form is compared.

The test objects were as follows: the composition obtained in Example 1 of the present invention; a mixture of the vortioxetine crystalline form (β crystalline form) and D-mannitol and povidone K30 were physically mixed. The weight ratio of 1:1:1, vortioxetine crystalline form (β crystalline form) was prepared according to patent CN101472906 Example 4c.

Determination of Apparent Solubility: A mixture of the composition obtained in Example 1 of the present invention and the vortioxetine crystalline form, respectively, was weighed into two plugged Erlenmeyer flasks, diluted with a predetermined pH value, prepared as a supersaturated solution, tightly closed lid. Three samples were prepared in parallel in each pH dilution. Placed in 37° C.±0.5° C. shaking water bath shaker 12 h, made it fully dissolved in order to achieve saturation. The supernatant was filtered with 0.45 micron microporous filter while it was still hot, and after appropriate dilution, shaken, were injected into the liquid chromatograph. The external standard method was used to calculate the apparent solubilities of three parallel samples in this pH buffer, averaged.

Preparation of Various pH Diluents:

(1) pH=1.0 diluent: 9 ml of concentrated hydrochloric acid was diluted with water to 1000 ml to obtain the solution.

(2) pH=4.5 diluent: took 7.7 g of ammonium acetate, dissolved in 50 ml of water and added 6 ml of glacial acetic acid and appropriate amount of water to make 100 ml to obtain the solution.

(3) pH=6.8 diluent: took 0.2 mol/L potassium dihydrogen phosphate solution 250 ml, added 0.2 mol/L sodium hydroxide solution 118 ml, diluted with water to 1000 ml, shook to obtain the solution.

(4) pH=7.4 diluent: Took 1.36 g of monobasic potassium phosphate, added 79 ml of 0.1 mol/L sodium hydroxide solution, and diluted to 200 ml with water to obtain the solution.

The experimental results are shown in Table 3:

| | Apparent solubility (μg/mL) | |
|---|---|---|
| | The composition of | |
| | Example 1 of the present | A mixture of vortioxetine |
| Diluent pH | invention | crystalline forms |
| 1.0 | 2.21 | 0.80 |
| 4.5 | 4.18 | 2.42 |
| 6.8 | 0.14 | 0.080 |
| 7.4 | 0.090 | 0.052 |

Table 3 shows that at each pH, the apparent solubility of the composition of amorphous vortioxetine and D-mannitol, povidone K30 of the present invention was significantly higher than the apparent solubility of the vortioxetine crystalline form (β) mixture.

### Example 38

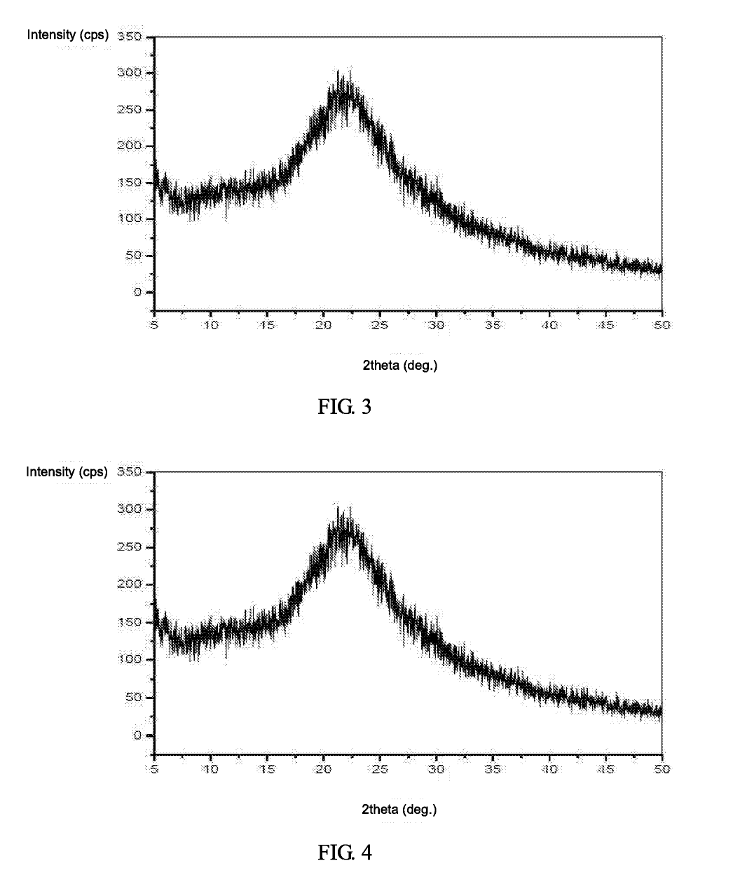

Vortioxetine hydrobromide (50 mg) and povidone K30 (30 mg) were added to methanol (900 μL) and heated to 60° C. to be stirred and clarified. Then Colloidal silica Aerosil 200 (30 mg) was added. The mixture was quickly cooled to −10° C. and a white solid precipitated, which was filtered and dried to give 102 mg of the amorphous vortioxetine hydrobromide and Povidone K30 and colloidal silica Aerosil 200 composition. The active ingredient load factor is 47.2%. The composition of the X-ray powder diffraction pattern is shown in FIG. 3. As can be seen from FIG. 3, X-ray powder diffraction pattern after subtracting the background of pharmaceutical excipients peak showed no vortioxetine hydrobromide crystal characteristic peak.

### Example 39

Vortioxetine (50 mg) and hydroxypropylmethylcellulose HPMC E3 (30 mg) were added to methanol (800 μl) and methylene chloride (800 μl), and the mixture was stirred and dissolved at 40° C., then colloidal silica Aerosil 200 (30 mg) was added. The above mixture was slowly concentrated to dryness in a rotary evaporator and further dried in vacuo to give a white solid, 110 mg of a combination of amorphous vortioxetine and hydroxypropylmethylcellulose HPMC E3 and colloidal silica Aerosil 200. The active ingredient loading rate was 45.4%. The X-ray powder diffraction pattern of the composition is shown in FIG. 4. The characteristic peak of the vortioxetine hydrobromide crystalline form is not seen after subtracting the background peak of the pharmaceutical excipient from the powder X-ray diffraction pattern.

### Example 40

Vortioxetine hydrobromide (2 g) and polyethylene glycol 8000 (1.2 g) were added to methanol (50 ml) and heated to 60° C. with stirring to dissolve. Magnesium aluminum silicate Neusilin UFL2 (0.6 g) was then added. The mixture was slowly concentrated to dryness in a rotary evaporator and further dried in vacuo to give a white solid which was further dried in vacuo to afford 3.8 g of a combination of amorphous vortioxetine hydrobromide with polyethylene glycol 8000 and Neusilin UFL2. The active ingredient loading was 52.6%. In the X-ray powder diffraction pattern of the composition, no characteristic peak of the vortioxetine hydrobromide crystalline form was obtained after subtracting the background peak of the pharmaceutical excipient.

### Example 41

Vortioxetine hydrobromide (1 g) and hydroxypropyl methylcellulose E50 (0.8 g) were added to water (10 ml) and methanol (10 ml), and the mixture was heated to 60° C. and stirred to clarify. Then Silica Syloid 244 FP (0.3 g) was added. The above mixture was slowly concentrated to dryness in a rotary evaporator and further dried in vacuo to give a white solid, ie, a 2.1 g of combination of amorphous vortioxetine hydrobromide and hydroxypropylmethylcellulose E50 and silica Syloid 244 FP, the active ingredient loading rate was 47.1%. In the X-ray powder diffraction pattern of the composition, no characteristic peak of the vortioxetine hydrobromide crystalline form was observed after subtracting the background peak of the pharmaceutical excipient.

### Example 42

Vortioxetine hydrobromide (5 g), urea (0.5 g) and povidone K30 (2.5 g) were added to methanol (200 ml) and heated to 60° C. with stirring to dissolve. Then, aluminum silicate Magnesium Neusilin UFL2 (0.6 g) was added. The above mixture was slowly concentrated to dryness on a rotary evaporator and further dried in vacuo to give a white solid, yielding 8 g of a composition of amorphous vortioxetine hydrobromide with urea, povidone K30 and Neusilin UFL2, the active ingredient loading rate was 62.5%. In the X-ray powder diffraction pattern of the composition, no characteristic peak of the vortioxetine crystalline form was found after subtracting the background peak of the pharmaceutical excipient.

### Example 43

Vortioxetine hydrobromide (1 g) and polyacrylic resin Eudragit L100 (0.8 g) were added to methanol (30 ml) and heated to 60° C. to dissolve them. Neusilin UFL2 (0.6 g) was then added. The mixture was rapidly cooled to 10° C., a white solid was precipitated, filtered and dried to give 2.3 g of a combination of amorphous vortioxetine hydrobromide and polyacrylic acid resin Eudragit L100 and Neusilin UFL2. The load rate was 42.1%. In the X-ray powder diffraction pattern of the composition, no characteristic peak of the vortioxetine hydrobromide crystalline form was observed after subtracting the background peak of the pharmaceutical excipient.

### Example 44

Vortioxetine hydrobromide (1 g) and polyacrylic resin Eudragit L100 (0.6 g) were added to methanol (30 ml), heated to 60° C., stirred and dissolved, then added Syloid 72FP (0.4 g). The mixture was rapidly cooled down to 10° C., a white solid was precipitated, filtered and dried to obtain 1.9 g of a composition of amorphous vortioxetine hydrobromide and polyacrylic acid resin Eudragit L100 and silica Syloid 72 FP. The load of the active ingredient was 50.4%. In the X-ray powder diffraction pattern of the composition, no characteristic peak of the vortioxetine hydrobromide crystalline form was observed after subtracting the background peak of the pharmaceutical excipient.

### Example 45

Vortioxetine hydrobromide (1 g) and povidone K30 (0.5 g) were added to methylene chloride (20 ml), heated to 40° C., stirred and dissolved, and then added with aluminum magnesium silicate Neusilin UFL2 (0.6 g). The mixture was rapidly added to n-heptane (200 ml), a white solid precipitated, which was filtered and dried to give 2.0 g of a combination of amorphous vortioxetine hydrobromide and Povidone K30 and Neusilin UFL2. The active ingredient loading rate was 48.5%. In the X-ray powder diffraction pattern of the composition, no characteristic peak of the vortioxetine hydrobromide crystalline form was observed after subtracting the background peak of the pharmaceutical excipient.

### Example 46

Vortioxetine hydrobromide (1 g) and povidone K30 (0.6 g) were added to methylene chloride (20 ml), heated to 40° C., stirred and dissolved, and colloidal silica Aerosil 380 (0.3 g) was added. The mixture was rapidly added to n-heptane (200 mL) and a white solid precipitated, which was filtered and dried to afford the composition 1.84 g of amorphous vortioxetine hydrobromide with Povidone K30 and colloidal silica Aerosil 380 G, the active ingredient loading rate was 53.1%. In the X-ray powder diffraction pattern of the composition, no characteristic peak of the vortioxetine hydrobromide crystalline form was observed after subtracting the background peak of the pharmaceutical excipient.

### Example 47

Vortioxetine hydrobromide (1 g) and povidone K30 (0.7 g) were added to methylene chloride (20 ml), and the mixture was heated to 40° C. to be stirred and dissolved. Then, Syloid 244 FP (0.3 g) was added. The mixture was rapidly added to n-heptane (200 mL), a white solid precipitated, which was filtered and dried to give 1.92 g of a combination of amorphous vortioxetine hydrobromide and Povidone K30 and Silica Syloid 244 FP. The active ingredient loading rate was 50.8%. In the X-ray powder diffraction pattern of the composition, no characteristic peak of the vortioxetine hydrobromide crystalline form was observed after subtracting the background peak of the pharmaceutical excipient.

### Example 48

Vortioxetine hydrobromide (1 g) and povidone K30 (2 g) were added to methylene chloride (20 ml) and heated to 40° C. with stirring to dissolve. Silica Syloid 244 FP (1 g) was added. The mixture was rapidly added to n-heptane (200 mL), a white solid precipitated, which was filtered and dried to afford 3.8 g of the combination of amorphous vortioxetine hydrobromide and Povidone K30 and Silica Syloid 244 FP. The active ingredient loading rate was 25.1%. In the X-ray powder diffraction pattern of the composition, no characteristic peak of the vortioxetine hydrobromide crystalline form was observed after subtracting the background peak of the pharmaceutical excipient.

### Example 49

Vortioxetine hydrobromide (1 g) and povidone K30 (0.7 g) were added to methylene chloride (20 ml), heated to 40° C., stirred and dissolved, and magnesium oxide (0.5 g) was added. The mixture was rapidly added to n-heptane (200 ml) to precipitate a white solid, which was filtered and dried to give 2.08 g of amorphous vortioxetine hydrobromide and Povidone K30 and magnesium oxide composition. The load factor of the active ingredient was 47.1%. In the X-ray powder diffraction pattern of the composition, no characteristic peak of the vortioxetine hydrobromide crystalline form was observed after subtracting the background peak of the pharmaceutical excipient.

### Example 50

Vortioxetine hydrobromide (1 g) and hydroxypropylmethylcellulose HPMC E3 (0.8 g) were added to methylene chloride (20 ml), heated to 40° C., stirred and dissolved, and then zinc oxide (0.7 grams) was added. The above mixture was concentrated to dryness on a rotary evaporator and further dried to give 2.5 g of a combination of amorphous vortioxetine hydrobromide and HPMC E3 and zinc oxide with a loading of the active ingredient of 40%. In the X-ray powder diffraction pattern of the composition, no characteristic peak of the vortioxetine hydrobromide crystalline form was observed after subtracting the background peak of the pharmaceutical excipient.

### Example 51

Vortioxetine hydrobromide (1 g) and hydroxypropylmethylcellulose HPMC E3 (2 g) were added to methylene chloride (20 ml), heated to 40° C., stirred and dissolved, and then titanium dioxide (5 g) was added. The above mixture was concentrated to dryness on a rotary evaporator and further dried to afford 8 g of a combination of amorphous vortioxetine hydrobromide and HPMC E3 and titanium dioxide composition with a loading rate of active ingredient of 12.5%. In the X-ray powder diffraction pattern of the composition, no characteristic peak of the vortioxetine hydrobromide crystalline form was observed after subtracting the background peak of the pharmaceutical excipient.

### Example 52

Vortioxetine hydrobromide (1 g) and hydroxypropylmethylcellulose HPMC E3 (3 g) were added to methylene chloride (20 ml), heated to 40° C., stirred and dissolved, and then colloidal bis Silica Aerosil 380 (5 g). The above mixture was concentrated to dryness on a rotary evaporator and further dried to afford 9 g of a combination of amorphous vortioxetine hydrobromide and hydroxypropylmethylcellulose HPMC E3 and Aerosil 380 with a loading of active ingredient of 11.1%. In the X-ray powder diffraction pattern of the composition, no characteristic peak of the vortioxetine hydrobromide crystalline form was observed after subtracting the background peak of the pharmaceutical excipient.

### Example 53

Vortioxetine hydrobromide (1 g) and povidone K30 (3 g) were added to tetrahydrofuran (60 ml), and the mixture was heated to 60° C. for dissolution. The colloidal silica Aerosil 380 (5 g) was then added. The above mixture was concentrated to dryness on a rotary evaporator and further dried to give 9 g of a combination of the amorphous vortioxetine hydrobromide composition with Povidone K30 and Aerosil 380 with a loading of 11.1% active ingredient. In the X-ray powder diffraction pattern of the composition, no characteristic peak of the vortioxetine hydrobromide crystalline form was observed after subtracting the background peak of the pharmaceutical excipient.

### Example 54: Influencing Factors for Compositions of Amorphous Vortioxetine Hydrobromide, Povidone K30 and Colloidal Silica (Aerosil 200) Test

Materials: The combination of amorphous vortioxetine hydrobromide obtained in Example 38, Povidone K30 and colloidal silica (Aerosil 200), the results of which are shown in Table 4.

TABLE 4

| | | Condition | | | |
|---|---|---|---|---|---|
| | | Temperature | Temperature | | |
| | | 40° C. ± | 60° C. ± | Humidity | Humidity |
| Test items | time | 2° C. | 2° C. | 75% ± 5% | 90% ± 5% |
| Related | 0 d | 0.09 | | | |
| substances | 5 d | 0.10 | 0.11 | 0.09 | 0.10 |
| (total | 10 d | 0.12 | 0.14 | 0.10 | 0.11 |
| impurity %) | | | | | |

| | | |
|---|---|---|
| Form (X- | 0 d | After removing the background peaks of the |
| powder | | pharmaceutical excipients, there was |
| diffraction | | no characteristic peak of the vortioxetine |
| pattern) | | hydrobromide crystalline form |
| | 5 d | After removing the background peaks of the |
| | | pharmaceutical excipients, there was no |
| | | characteristic peak of the vortioxetine |
| | | hydrobromide crystalline form |
| | 10 d | After removing the background peaks of the |
| | | pharmaceutical excipients, there was no |
| | | characteristic peak of the vortioxetine |
| | | hydrobromide crystalline form |

Table 4 shows that the composition of amorphous vortioxetine hydrobromide, povidone K30, and colloidal silica (Aerosil 200) was allowed to stand for 10 days at high temperature and high humidity without any significant change in the substance of interest, no vortioxetine hydrobromide crystallized out.

### Example 55: Accelerated Test of Compositions of Amorphous Vortioxetine Hydrobromide, Povidone K30 and Colloidal Silica (Aerosil 200)

Materials: Composition of amorphous vortioxetine hydrobromide obtained in Example 38, povidone K30, and colloidal silica (Aerosil 200).

Experimental conditions: temperature 40° C.±2° C., humidity 75%±5%, the results shown in Table 5.

TABLE 5

| | | Condition |
|---|---|---|
| Test items | Time | Temperature 40° C. ± 2° C., Humidity 75% ± 5% |
| Related | 0 M | 0.09 |
| substances | 1 M | 0.10 |
| (total | 2 M | 0.11 |
| impurity %) | 3 M | 0.12 |
| | 6 M | 0.14 |
| Form (X- | 0 M | After removing the background peaks of the |
| powder | | pharmaceutical excipients, there was no |
| diffraction | | characteristic peak of the vortioxetine |
| pattern) | | hydrobromide crystalline form |
| | 1 M | After removing the background peaks of the |
| | | pharmaceutical excipients, there was no |
| | | characteristic peak of the vortioxetine hydrobromide |
| | | crystalline form |
| | 2 M | After removing the background peaks of the |
| | | pharmaceutical excipients, there was no |
| | | characteristic peak of the vortioxetine hydrobromide |
| | | crystalline form |
| | 3 M | After removing the background peaks of the |
| | | pharmaceutical excipients, there was no |
| | | characteristic peak of the vortioxetine hydrobromide |
| | | crystalline form |
| | 6 M | After removing the background peaks of the |
| | | pharmaceutical excipients, there was no |
| | | characteristic peak of the vortioxetine hydrobromide |
| | | crystalline form |

Table 5 shows that the composition of amorphous vortioxetine hydrobromide, povidone K30, and colloidal silica (Aerosil 200) of the present invention was allowed to stand for 6 months under an accelerated test condition without significant change, no vortioxetine hydrobromide crystallized out.

### Example 56: Determination of Apparent Solubility

The apparent solubility of the mixture of the composition of the invention and the vortioxetine crystalline form is compared.

The test objects were as follows: the composition obtained in Example 38 of the present invention; the mixture of the vortioxetine crystalline form (the β crystalline form) and povidone K30, the colloidal silica (Aerosil 200) were physically mixed at a weight ratio of 1:1:1, and the vortioxetine crystalline form (β crystalline form) was prepared according to the method of Example 4c of CN101472906.

Determination of Apparent Solubility: A mixture of the composition obtained in Example 1 of the present invention and the vortioxetine crystalline form, respectively, was weighed into two plugged Erlenmeyer flasks, diluted with a predetermined pH value, prepared as a supersaturated solution, tightly closed lid. Three samples were prepared in parallel in each pH dilution. Placed in 37° C.±0.5° C. shaking water bath shaker 12 h, made it fully dissolved to reach saturation. The supernatant was filtered with 0.45 micron microporous filter while it was hot, and appropriately diluted, shaken, were injected into the liquid chromatograph. The external standard method was used to calculate the apparent solubilities of three parallel samples in this pH buffer, averaged.

Preparation of Various pH Diluents:

(1) pH=1.0 diluent: 9 ml of concentrated hydrochloric acid was diluted with water to 1000 ml to obtain the solution.

(2) pH=4.5 diluent: took 7.7 g of ammonium acetate, dissolved in 50 ml of water and added 6 ml of glacial acetic acid and appropriate amount of water to make 100 ml to obtain the solution.

(3) pH=6.8 diluent: took 0.2 mol/L potassium dihydrogen phosphate solution 250 ml, added 0.2 mol/L sodium hydroxide solution 118 ml, diluted with water to 1000 ml, mixed well to obtain the solution.

(4) pH=7.4 diluent: took 1.36 g of monobasic potassium phosphate, added 79 ml of 0.1 mol/L sodium hydroxide solution, and diluted to 200 ml with water to obtain the solution.

The experimental results are shown in Table 6:

TABLE 6

Apparent solubility (μg/mL)

| | Invention Example 38 | A mixture of vortioxetine |
|---|---|---|
| Diluent pH | Composition | crystalline forms |
| 1.0 | 2.21 | 0.72 |
| 4.5 | 4.58 | 2.18 |
| 6.8 | 0.15 | 0.070 |
| 7.4 | 0.081 | 0.042 |

Table 6 shows that at each pH, the apparent solubility of the combination of amorphous vortioxetine and D-mannitol, Povidone K30 of the present invention was significantly higher than the apparent solubility of the vortioxetine crystalline form (β crystalline form) mixture.

### Example 57

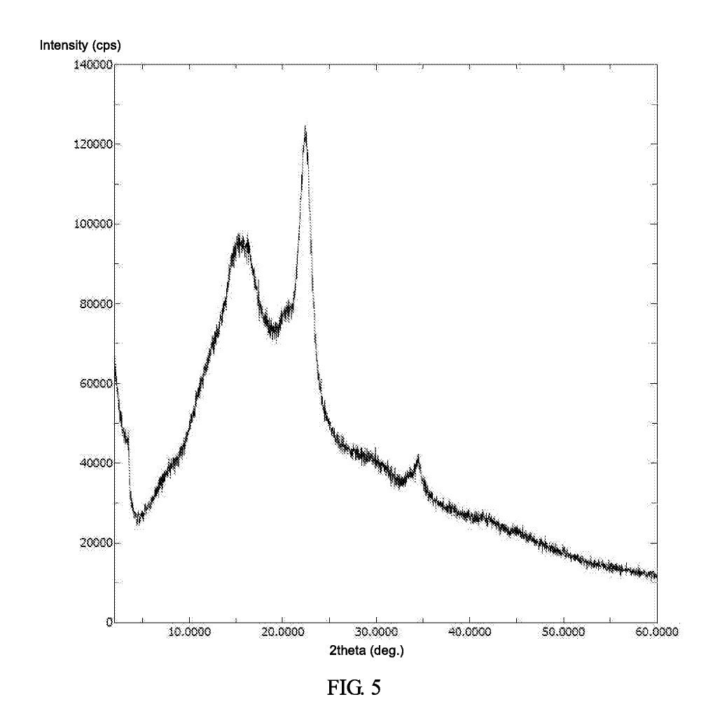

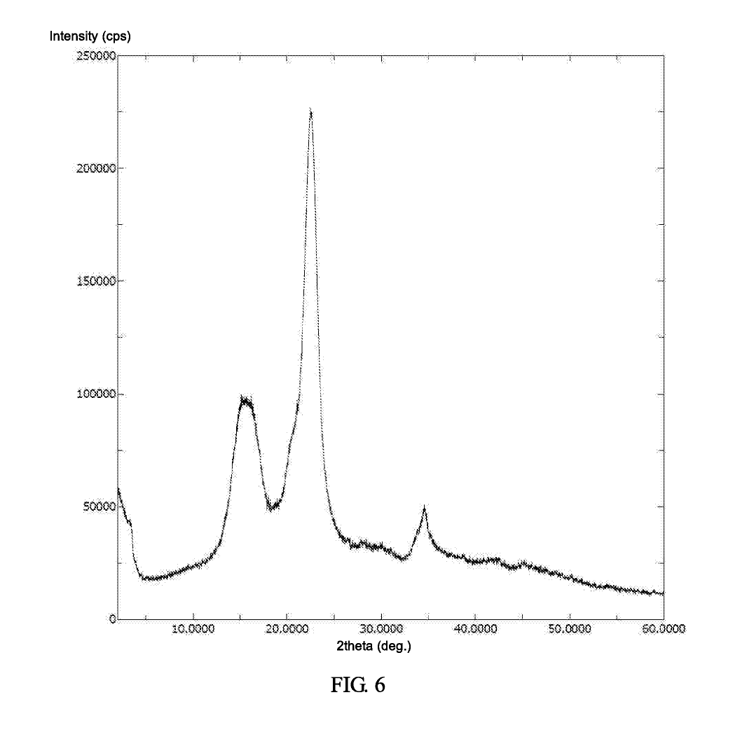

Vortioxetine hydrobromide (50 mg) and povidone K30 (30 mg) were added to methanol (900 μl) and heated to 60° C. to dissolve the mixture. Colloidal silica Aerosil 200 (30 mg) and microcrystalline cellulose (20 mg) were then added. The mixture was rapidly cooled to −10° C., a white solid precipitated, which was filtered and dried to give 121 mg of a composition of amorphous vortioxetine hydrobromide, povidone K30, microcrystalline cellulose and colloidal silica Aerosil 200, the active ingredient loading was 38.2%. The composition of the X-ray powder diffraction pattern shown in FIG. 5, it can be seen from FIG. 5, X-ray powder diffraction pattern after subtracting the background of the pharmaceutical excipients peak showed no vortioxetine hydrobromide crystalline form characteristic peaks, microcrystalline cellulose X-ray powder diffraction pattern shown in FIG. 6.

### Example 58

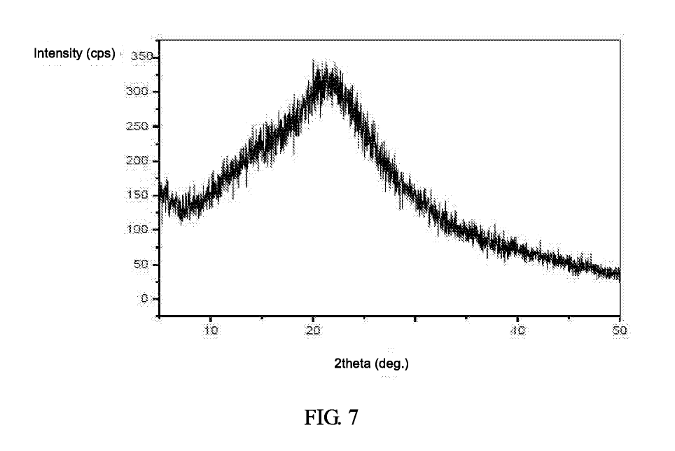

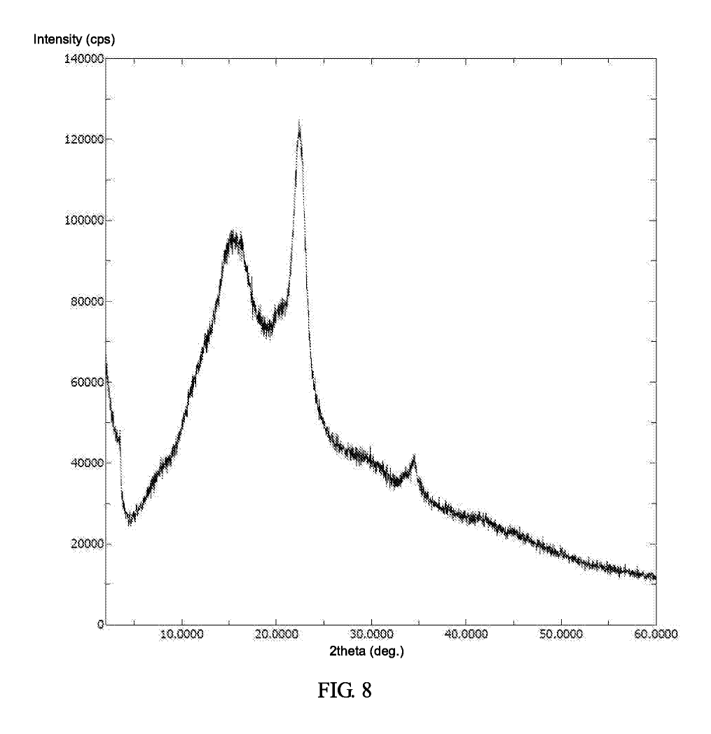

Vortioxetine (50 mg) and hydroxypropylmethylcellulose HPMC E3 (30 mg) were added to methanol (800 μl) and methylene chloride (800 μl), and the mixture was stirred and dissolved at 40° C., then added Colloidal silica Aerosil 200 (30 mg) and mannitol (50 mg). The above mixture was slowly concentrated to dryness in a rotary evaporator and further dried in vacuo to give a white solid, ie, 160 mg of a combination of amorphous vortioxetine, HPMC E3, mannitol and colloidal silica Aerosil 200, and the active ingredient loading rate was 31.2%. The X-ray powder diffraction pattern of the composition is shown in FIG. 7, and it can be seen from FIG. 7, X-ray powder diffraction pattern after subtracting the background of the pharmaceutical excipients peak showed no vortioxetine hydrobromide crystalline form characteristic peaks.

### Example 59

Vortioxetine hydrobromide (2 g) and polyethylene glycol 8000 (1.2 g) were added to methanol (50 ml) and heated to 60° C. with stirring to dissolve. Magnesium aluminum silicate Neusilin UFL2 (0.6 g) and lactose (2 g) were then added. The mixture was slowly concentrated to dryness on a rotary evaporator and further dried in vacuo to give a white solid which was further dried in vacuo to afford 5.8 g of a combination of amorphous vortioxetine hydrobromide, polyethylene glycol 8000, lactose, and magnesium aluminum silicate Neusilin UFL2, and the active ingredient loading was 34.4%. In the X-ray powder diffraction pattern of the composition, no characteristic peak of the vortioxetine hydrobromide crystalline form was observed after subtracting the background peak of the pharmaceutical excipient.

### Example 60

Vortioxetine hydrobromide (1 g) and hydroxypropyl methylcellulose E50 (0.8 g) were added to water (10 ml) and methanol (20 ml), heated to 60° C. to stir and dissolve, Silica Syloid 244 FP (0.3 g) and microcrystalline cellulose (0.5 g) were then added. The above mixture was slowly concentrated to dryness in a rotary evaporator and further dried in vacuo to give a white solid, ie, 2.8 g of a combination of amorphous vortioxetine hydrobromide and hydroxypropylmethylcellulose E50 and silica Syloid 244 FP, the active ingredient loading was 35.7%. In the X-ray powder diffraction pattern of the composition, no characteristic peak of the vortioxetine hydrobromide crystalline form was observed after subtracting the background peak of the pharmaceutical excipient.

### Example 61

Vortioxetine hydrobromide (5 g), urea (0.5 g) and povidone K30 (2.5 g) were added to methanol (200 ml) and heated to 60° C. with stirring to dissolve. Then, aluminum silicate Magnesium Neusilin UFL2 (0.6 g) and mannitol (5 g) were added. The mixture was slowly concentrated to dryness in a rotary evaporator and further dried in vacuo to give a white solid, ie, 14.1 g of a combination of amorphous vortioxetine hydrobromide and urea, povidone K30, mannitol and Neusilin UFL2. The active ingredient loading was 35.4%. In the X-ray powder diffraction pattern of the composition, no characteristic peak of the vortioxetine crystalline form was found after subtracting the background peak of the pharmaceutical excipient.

### Example 62

Vortioxetine hydrobromide (1 g) and polyacrylic resin Eudragit L100 (0.8 g) were added to methanol (30 ml) and heated to 60° C. to dissolve them. Neusilin UFL2 (0.6 g) and sodium carboxymethyl starch (1 g) were then added. The mixture was rapidly cooled to 10° C., a white solid was precipitated, filtered and dried to give 3.2 g of a composition of amorphous vortioxetine hydrobromide and polyacrylic resin Eudragit L100, sodium carboxymethyl starch and Neusilin UFL2, the active ingredient loading rate was 30.1%. In the X-ray powder diffraction pattern of the composition, no characteristic peak of the vortioxetine hydrobromide crystalline form was observed after subtracting the background peak of the pharmaceutical excipient.

### Example 63

Vortioxetine hydrobromide (1 g) and polyacrylic resin Eudragit L100 (0.6 g) were added to methanol (30 ml), heated to 60° C., stirred and dissolved, then Syloid 72FP (0.4 g) and mannitol (0.1 g) were added. The mixture was rapidly cooled to 10° C., a white solid was precipitated, filtered and dried to afford 2.0 g of a combination of amorphous vortioxetine hydrobromide and polyacrylic resin Eudragit L100 and silica Syloid 72 FP. The loading rate of the active ingredient was 50.8%. In the X-ray powder diffraction pattern of the composition, no characteristic peak of the vortioxetine hydrobromide crystalline form was observed after subtracting the background peak of the pharmaceutical excipient.

### Example 64

Vortioxetine hydrobromide (1 g) and povidone K30 (0.5 g) were added to methylene chloride (25 ml), heated to 40° C., stirred and dissolved, and then added with aluminum magnesium silicate Neusilin UFL2 (0.6 g). The mixture was rapidly added to a mixture of n-heptane (200 ml) and microcrystalline cellulose (2 g) to precipitate a white solid, which was filtered and dried to give 4.0 g of a combination of amorphous vortioxetine hydrobromide, Povidone K30, microcrystalline cellulose and Neusilin UFL2 magnesium aluminosilicate, with a loading of 24.5% active ingredient. In the X-ray powder diffraction pattern of the composition, no characteristic peak of the vortioxetine hydrobromide crystalline form was observed after subtracting the background peak of the pharmaceutical excipient.

### Example 65

Vortioxetine hydrobromide (1 g) and povidone K30 (0.6 g) were added to methylene chloride (20 ml), heated to 40° C., stirred and dissolved, and colloidal silica Aerosil 380 (0.3 g) was then added. The mixture was rapidly added to a mixture of n-heptane (200 ml) and microcrystalline cellulose (2 g) to precipitate a white solid, which was filtered and dried to give 3.9 g of a composition of amorphous vortioxetine hydrobromide, povidone K30, and colloidal silica Aerosil 380 with a loading of 24.3% active ingredient. In the X-ray powder diffraction pattern of the composition, no characteristic peak of the vortioxetine hydrobromide crystalline form was observed after subtracting the background peak of the pharmaceutical excipient.

### Example 66

Vortioxetine hydrobromide (1 g) and povidone K30 (0.7 g) were added to methylene chloride (20 ml), and the mixture was heated to 40° C. to be stirred and dissolved. Then, Syloid 244 FP (0.3 g) was added. The above mixture was rapidly added to a mixture of n-heptane (200 ml) and croscarmellose sodium (2 g) to precipitate a white solid, which was filtered and dried to give 3.9 g of a combination of amorphous vortioxetine hydrobromide, povidone K30, croscarmellose sodium and silica Syloid 244 FP, with a loading of 24.6% active ingredient. In the X-ray powder diffraction pattern of the composition, no characteristic peak of the vortioxetine hydrobromide crystalline form was observed after subtracting the background peak of the pharmaceutical excipient.

### Example 67

Vortioxetine hydrobromide (1 g) and povidone K30 (2 g) were added to methylene chloride (20 ml) and heated to 40° C. with stirring to dissolve. Silica Syloid 244 FP (1 g) and croscarmellose sodium (0.5 g) were then added. The mixture was slowly concentrated to dryness in a rotary evaporator and further dried in vacuo to give white solid, ie, a 4.5 grams of a composition of amorphous vortioxetine hydrobromide, Povidone K30, croscarmellose sodium and silica Syloid 244 FP, the active ingredient loading rate was 22.2%. In the X-ray powder diffraction pattern of the composition, no characteristic peak of the vortioxetine hydrobromide crystalline form was observed after subtracting the background peak of the pharmaceutical excipient.

### Example 68

Vortioxetine hydrobromide (1 g) and povidone K30 (0.7 g) were added to methylene chloride (20 ml), heated to 40° C., stirred and dissolved, and then magnesium oxide (0.5 g) and Mannitol (0.5 g) were added. The above mixture was slowly concentrated to dryness in a rotary evaporator and further dried in vacuo to give a white solid, ie, 2.7 g of the composition of amorphous vortioxetine hydrobromide, Povidone K30, mannitol and magnesium oxide. The loading rate of the active components was 37.0%. In the X-ray powder diffraction pattern of the composition, no characteristic peak of the vortioxetine hydrobromide crystalline form was observed after subtracting the background peak of the pharmaceutical excipient.

### Example 69

Vortioxetine hydrobromide (1 g) and hydroxypropylmethylcellulose HPMC E3 (0.8 g) were added to methylene chloride (25 ml) and heated to 40° C. with stirring to dissolve and then zinc oxide (0.7 g) and microcrystalline cellulose (1 g) were added. The above mixture was concentrated to dryness on a rotary evaporator and further dried to give 3.5 g of a combination of amorphous vortioxetine hydrobromide, hydroxypropylmethylcellulose HPMC E3, microcrystalline cellulose and zinc oxide, and the active component loading rate of the composition was 28.5%. In the X-ray powder diffraction pattern of the composition, no characteristic peak of the vortioxetine hydrobromide crystalline form was observed after subtracting the background peak of the pharmaceutical excipient.

### Example 70

Vortioxetine hydrobromide (1 g) and hydroxypropylmethylcellulose HPMC E3 (2 g) were added to methylene chloride (30 ml) and heated to 40° C. with stirring to dissolve. Titanium dioxide (5 g) and microcrystalline cellulose (2 g) were then added. The above mixture was concentrated to dryness on a rotary evaporator and further dried to give 10 g of a combination of amorphous vortioxetine hydrobromide, hydroxypropylmethylcellulose HPMC E3, microcrystalline cellulose and titanium dioxide, and the active ingredient loading rate was 10.0%. In the X-ray powder diffraction pattern of the composition, no characteristic peak of the vortioxetine hydrobromide crystalline form was observed after subtracting the background peak of the pharmaceutical excipient.

### Example 71

Vortioxetine hydrobromide (1 g) and hydroxypropylmethylcellulose HPMC E3 (3 g) were added to methylene chloride (20 ml) and heated to 40° C. with stirring to dissolve. Then, colloidal bis Silica Aerosil 380 (5 g) and lactose (1 g) were added. The above mixture was concentrated to dryness on a rotary evaporator and further dried to give 10 g of a composition of amorphous vortioxetine hydrobromide, hydroxypropylmethylcellulose HPMC E3, lactose and Aerosil 380, loading rate of the active ingredient was 10.0%. In the X-ray powder diffraction pattern of the composition, no characteristic peak of the vortioxetine hydrobromide crystalline form was observed after subtracting the background peak of the pharmaceutical excipient.

### Example 72

Vortioxetine hydrobromide (1 g) and povidone K30 (3 g) were added to tetrahydrofuran (60 ml), and the mixture was heated to 60° C. for dissolution. The colloidal silica Aerosil 380 (5 g) and microcrystalline cellulose (1 g) then added. The above mixture was concentrated to dryness on a rotary evaporator and further dried to give 10 g of a combination of amorphous vortioxetine hydrobromide, Povidone K30, microcrystalline cellulose and Aerosil 380 with a loading of the active ingredient of 10.0%. In the X-ray powder diffraction pattern of the composition, no characteristic peak of the vortioxetine hydrobromide crystalline form was observed after subtracting the background peak of the pharmaceutical excipient.

### Example 73

Vortioxetine hydrobromide (1 g) and povidone K30 (3 g) were added to tetrahydrofuran (60 ml), and the mixture was heated to 60° C. for dissolution. The colloidal silica Aerosil 380 (5 g) and microcrystalline cellulose (1 g) were then added. The above mixture was concentrated to dryness on a rotary evaporator and further dried to give 10 g of a combination of amorphous vortioxetine hydrobromide, Povidone K30, microcrystalline cellulose and Aerosil 380 with a loading of the active ingredient of 10.0%. In the X-ray powder diffraction pattern of the composition, no characteristic peak of the vortioxetine hydrobromide crystalline form was observed after subtracting the background peak of the pharmaceutical excipient.

### Example 74

Vortioxetine hydrobromide (1 g) and hydroxypropylmethylcellulose HPMC E3 (0.8 g) were added to methanol (30 ml) and heated to 60° C. to be stirred and dissolved. Colloidal silica Aerosil 200 (0.5 g) was then added. The mixture was spray dried in a fluid bed and loaded onto lactose (3 g) to give 4.7 g of a combination of amorphous vortioxetine hydrobromide, povidone K30, lactose and Aerosil 200, the load rate of active ingredient was 20.5%. In the X-ray powder diffraction pattern of the composition, no characteristic peak of the vortioxetine hydrobromide crystalline form was observed after subtracting the background peak of the pharmaceutical excipient.

### Example 75: Influencing Factors for Compositions of Amorphous Vortioxetine Hydrobromide, Povidone K30, Microcrystalline Cellulose and Colloidal Silica (Aerosil 200) Test

Materials: Composition of amorphous vortioxetine hydrobromide obtained in Example 57, povidone K30, microcrystalline cellulose and colloidal silica (Aerosil 200), the results are shown in Table 7.

TABLE 7

| | | Condition | | | |
|---|---|---|---|---|---|
| | | Temperature | | | |
| | | 40° C. ± | Temperature | Humidity | Humidity |
| Test items | Time | 2° C. | 60° C. ± 2° C. | 75% ± 5% | 90% ± 5% |
| Related | 0 d | 0.09 | | | |
| substances | 5 d | 0.10 | 0.11 | 0.09 | 0.10 |
| (total | 10 d | 0.12 | 0.13 | 0.10 | 0.11 |
| impurit %) | | | | | |

| | | |
|---|---|---|
| Form (X- | 0 d | After removing the background peaks of the |
| powder | | pharmaceutical excipients, there was no |
| diffraction | | characteristic peak of the vortioxetine |
| pattern) | | hydrobromide crystalline form |
| | 5 d | After removing the background peaks of the |
| | | pharmaceutical excipients, there was no |
| | | characteristic peak of the vortioxetine |
| | | hydrobromide crystalline form |
| | 10 d | After removing the background peaks of the |
| | | pharmaceutical excipients, there was no |
| | | characteristic peak of the vortioxetine |
| | | hydrobromide crystalline form |

Table 7 shows that the combination of amorphous vortioxetine hydrobromide, Povidone K30, microcrystalline cellulose, and colloidal silica (Aerosil 200) was allowed to stand for 10 days under conditions of high temperature and high humidity, there was no significant change in related substance, there was no vortioxetine hydrobromide crystallization.

### Example 76: Accelerated Test of Compositions of Amorphous Vortioxetine Hydrobromide, Povidone K30, Microcrystalline Cellulose and Colloidal Silica (Aerosil 200)

Materials: Composition of amorphous vortioxetine hydrobromide obtained in Example 57, povidone K30, microcrystalline cellulose and colloidal silica (Aerosil 200) Experimental conditions: Temperature 40° C.±2° C., humidity 75%±5%.

TABLE 8

| | | Condition |
|---|---|---|
| Test items | Time | Temperature 40° C. ± 2° C., Humidity 75% ± 5% |
| Related | 0 M | 0.09 |
| substances | 1 M | 0.10 |
| (total | 2 M | 0.11 |
| impurity %) | 3 M | 0.12 |
| | 6 M | 0.14 |
| Form (X- | 0 M | After removing the background peaks of the |
| powder | | pharmaceutical excipients, there was no |
| diffraction | | characteristic peak of the vortioxetine |
| pattern) | | hydrobromide crystalline form |
| | 1 M | After removing the background peaks of the |
| | | pharmaceutical excipients, there was no |
| | | characteristic peak of the vortioxetine |
| | | hydrobromide crystalline form |
| | 2 M | After removing the background peaks of the |
| | | pharmaceutical excipients, there was no |
| | | characteristic peak of the vortioxetine |
| | | hydrobromide crystalline form |
| | 3 M | After removing the background peaks of the |
| | | pharmaceutical excipients, there was no |
| | | characteristic peak of the vortioxetine |
| | | hydrobromide crystalline form |
| | 6 M | After removing the background peaks of the |
| | | pharmaceutical excipients, there was no |
| | | characteristic peak of the vortioxetine |
| | | hydrobromide crystalline form |

Table 8 shows that the combination of amorphous vortioxetine hydrobromide, Povidone K30, microcrystalline cellulose, and colloidal silica (Aerosil 200) was placed under accelerated test conditions for 6 months, and the relevant substance had no significant change, no vortioxetine hydrobromide crystallized out.

### Example 77: Apparent Solubility was Determined

The apparent solubility of the mixture of the composition of the invention and the vortioxetine crystalline form is compared.

The test objects were respectively the composition obtained in Example 57 of the present invention, the mixture of the vortioxetine crystal polymorphs (β crystalline form) with Povidone K30, microcrystalline cellulose and colloidal crystals Silica (Aerosil 200) were physically mixed in a weight ratio of 1:0.6:0.6:0.4. The vortioxetine crystalline form (β crystalline form) was prepared according to the method of Example 4c of CN101472906.

Determination of Apparent Solubility: A mixture of the composition obtained in Example 57 of the present invention and the vortioxetine crystalline form, respectively, was weighed into two plugged Erlenmeyer flasks, diluted with a predetermined pH, prepared as a supersaturated solution, tightly closed lid. Three samples were prepared in parallel in each pH dilution. Placed in 37° C.±0.5° C. shaking water bath shaker 12 h, made it fully dissolved to reach saturation. The supernatant was filtered with 0.45 micron microporous filter while it was hot, and appropriately diluted, shaken, were injected into the liquid chromatograph. The external standard method was used to calculate the apparent solubilities of three parallel samples in this pH buffer, averaged.

Preparation of Various pH Diluents:

(1) pH=1.0 diluent: 9 ml of concentrated hydrochloric acid was diluted with water to 1000 ml to obtain the solution.

(2) pH=2.0 diluent: A liquid: Took 16.6 ml of phosphoric acid, added water to 100 ml and shook well. B liquid: Took 71.63 grams of disodium hydrogen phosphate, added water to dissolve into 1000 ml. Took the above A liquid 72.5 ml and 27.5 ml of liquid B mixed, shook to obtain the solution.

(3) pH=3.0 diluent: Took 50 ml of glacial acetic acid and added 800 ml of water to adjust pH value to 3.0 with lithium hydroxide and diluted to 1000 ml with water to obtain the solution.

(4) pH=4.5 diluent: Took 7.7 g of ammonium acetate, dissolved in 50 ml of water and added 6 ml of glacial acetic acid and appropriate amount of water to 100 ml to obtain the solution.

(5) pH=5.6 diluent: Took 10 g of potassium hydrogen phthalate, added 900 ml of water and stirred to dissolve. Adjusted the pH value to 5.6 with sodium hydroxide solution (dilute hydrochloric acid if necessary) and diluted with water to 1000 ml, mixed to obtain the solution.

(6) pH=6.8 diluent: Took 0.2 mol/L potassium dihydrogen phosphate solution 250 ml, added 0.2 mol/L sodium hydroxide solution 118 ml, diluted with water to 1000 ml, shook to obtain the solution.

(7) pH=7.4 diluent: Took 1.36 g of monobasic potassium phosphate, added 79 ml of 0.1 mol/L sodium hydroxide solution, diluted to 200 ml with water to obtain the solution.

The experimental results are shown in Table 9:

TABLE 9

Apparent solubility (μg/mL)

| | Invention Example 57 | A mixture of vortioxetine |
|---|---|---|
| Diluent pH | Composition | crystalline forms |
| 1.0 | 2.08 | 0.82 |
| 4.5 | 3.92 | 2.13 |
| 5.6 | 0.14 | 0.072 |
| 7.4 | 0.076 | 0.052 |

Table 9 shows that at each pH, the apparent solubility of the composition of amorphous vortioxetine and D-mannitol, povidone K30 of the present invention was significantly higher than the apparent solubility of the vortioxetine crystalline form (β crystalline form) mixture.

The medicinal composition of amorphous vortioxetine hydrobromide of the invention obviously increases the dissolution rate and more facilitates the improvement of the bioavailability of the medicament so that the medicament can better exert the therapeutic effect of clinical diseases. The amorphous type, under the accelerated test conditions (40° C.±2° C., humidity 75%±5%), can maintain good physical stability and chemical stability.

1. A pharmaceutical composition comprising vortioxetine, or a pharmaceutically acceptable salt thereof, and two or more types of pharmaceutical excipients, wherein the weight ratio of vortioxetine or pharmaceutically acceptable salt thereof to total amount of the pharmaceutical excipients is 1:0.1˜100, wherein the vortioxetine or pharmaceutically acceptable salt thereof in the pharmaceutical composition is in an amorphous form, wherein in X-ray powder diffraction pattern of the composition, no characteristic peaks of crystalline vortioxetine or pharmaceutically acceptable salt thereof are present after deducting background peaks of the pharmaceutical excipients.

2. The pharmaceutical composition of claim 1, wherein the pharmaceutical excipients are selected from the group consisting of excipients, propellants, solubilizers, cosolvents, emulsifiers, colorants, binding agents, disintegrators, fillers, lubricants, wetting agents, tonicity adjusting agents, stabilizers, glidants, flavoring agents, preservatives, suspending agents, coating materials, fragrances, anti-adhesives, integrators, penetration enhancers, pH adjusters, buffers, plasticizers, surfactants, foaming agents, defoamers, thickeners, inclusion agents, humectants, absorbents, diluents, flocculants and anti-flocculants, antioxidants, adsorbents, filter aids, and release blockers.

3. The pharmaceutical composition of claim 1, wherein at least one of the pharmaceutical excipients is selected from hydroxypropylmethylcellulose, hydroxypropylcellulose, povidone, polyethylene glycol, ethylcellulose, liposomes, methacrylic acid copolymers, polyvinyl acetate, carboxymethylethylcellulose, carboxymethylcellulose phthalate, hydroxyethylcellulose methyl cellulose phthalate, hydroxypropylmethyl cellulose acetate succinate, polyacrylic resin, carbopol, alginate, carrageenan, carboxypolactone, gums, polyvinyl alcohol, pregelatinized starch, cross-linked starch, sodium carboxymethyl starch, dextrin, polyethylene oxide, chitosan, collagen, cyclodextrin, lactose, galactose, D-mannitol, Sorbitol, xylitol, urea, citric acid, tartaric acid, fumaric acid, maleic acid, and succinic acid.

4. The pharmaceutical composition of claim 1, wherein the vortioxetine is in the form of a hydrobromide salt, the pharmaceutical excipients comprise an organic carrier and an adsorbent, the vortioxetine hydrobromide forms a solid dispersion with an organic carrier, and the solid dispersion and the adsorbent form a composition; wherein the weight ratio of vortioxetine hydrobromide to the organic carrier is 1:0.1˜10, and the weight ratio of vortioxetine hydrobromide to the adsorbent is 1:0.1˜10; and wherein the vortioxetine hydrobromide in the composition is in an amorphous form, and X-ray powder diffraction pattern of the composition contains no characteristic peaks of vortioxetine hydrobromide crystals after deducting background peaks of the pharmaceutical excipients.

5. The pharmaceutical composition of claim 1, wherein the vortioxetine is in the form of a hydrobromide salt, the pharmaceutical excipients comprise an organic carrier, an adsorbent and a pharmaceutical formulation excipient; wherein the vortioxetine hydrobromide salt forms a solid dispersion with the organic carrier, and the solid dispersion forms a composition with the adsorbent and the pharmaceutical formulation excipient; wherein the weight of vortioxetine hydrobromide salt is 20%˜80% of the total weight of the solid dispersion, the weight of the adsorbent is 0.1%˜100% of the weight of the solid dispersion, and the weight of the pharmaceutical formulation excipient is 0.1%˜200% of the weight of the solid dispersion; and wherein the vortioxetine hydrobromide salt is amorphous, and X-ray powder diffraction pattern of the composition contains no characteristic peaks of vortioxetine hydrobromide crystals after deducting background peaks of the organic carrier, the adsorbent and the pharmaceutical formulation excipient.

6. The pharmaceutical composition of claim 5, wherein the adsorbent is selected from at least one of silica, aluminum oxide, titanium dioxide, magnesium oxide, calcium carbonate and zinc oxide.

7. The pharmaceutical composition of claim 4, wherein the organic carrier is selected from the group consisting of pharmaceutically acceptable small molecule organic compounds, polymers and copolymers.

8. The pharmaceutical composition of claim 4, wherein the organic carrier is selected from the group consisting of hydroxypropylmethylcellulose, hydroxypropylcellulose, povidone, polyethylene glycol, ethylcellulose, liposomes, methacrylic acid copolymers, polyvinyl acetate, carboxymethylethylcellulose, carboxymethylcellulose phthalate, hydroxypropyl methyl cellulose phthalate, hydroxypropyl methylcellulose acetate succinate, polyacrylic resin, carbopol, alginate, carrageenan, carboxylactone, gums, polyvinyl alcohol, pre-gelatinized starch, cross-linked starch, sodium carboxymethyl starch, dextrin, polyethylene oxide, chitosan, chitosan, collagen, cyclodextrin, lactose, galactose, D-mannitol, alcohol, xylitol, urea, citric acid, tartaric acid, fumaric acid, maleic acid, and succinic acid.

9. The pharmaceutical composition of claim 5, wherein the pharmaceutical formulation excipient is selected from the group consisting of excipients, propellants, solubilizers, emulsifiers, colorants, binders, disintegrants, fillers, lubricants, wetting agents, tonicity adjusting agents, stabilizers, glidants, flavoring agents, preservatives, suspending agents, coating materials, fragrances, anti-binders, integrating agents, penetration accelerators, pH adjusters, buffers, plasticizers, surfactants, blowing agents, defoamers, thickeners, clathrates, humectants, absorbents, dilution agents, flocculants and anti-flocculants, antioxidants, filter aids, and release retardants.

10. A method for preparing a pharmaceutical composition of claim 1, comprising the following steps:
1) mixing vortioxetine or a pharmaceutically acceptable salt thereof with pharmaceutical excipients, heating to melt the pharmaceutical excipients; wherein the weight ratio of vortioxetine or its pharmaceutically acceptable salt to the total pharmaceutical excipients is 1:0.1˜100;
2) cooling after mixing uniformly, pulverizing the resultant mixture to obtain a composition of amorphous vortioxetine or the pharmaceutically acceptable salt thereof and the pharmaceutical excipients.

11. The method of claim 10, wherein at least one of the pharmaceutical excipients is selected from the group consisting of self-excipients, propellants, solubilizers, cosolvents, emulsifiers, colorants, binders, disintegrants, fillers, lubricants, wetting agents, tonicity adjusting agents, stabilizers, glidants, flavoring agents, preservatives, suspending agents, coating materials, fragrances, anti-binders, integrators, penetration enhancers, pH adjusters, buffers, plasticizers, surfactants, foaming agents, defoamers, thickeners, humectants, absorbents, diluents, flocculants and deflocculants, antioxidants, adsorbents, filter aids, and release retardants.

12. The method of claim 10, wherein at least one of the pharmaceutical excipients is selected from the group consisting of hydroxypropylmethylcellulose, hydroxypropyl cellulose, povidone, polyethylene glycol, ethylcellulose, liposomes, methacrylic acid copolymer, polyvinyl acetate, carboxymethylethylcellulose, carboxymethylcellulose phthalate hydroxypropylmethylcellulose phthalate, hydroxypropylmethylcellulose acetate succinate, polyacrylic resin, carbopol, alginate, carrageenan, carboxypolactone, gums, poly pregelatinized starch, cross-linked starch, sodium carboxymethyl starch, dextrin, polyethylene oxide, chitosan, chitosan, collagen, cyclodextrin, lactose, galactose, D-mannitol, sorbitol, xylitol, urea, citric acid, tartaric acid, fumaric acid, maleic acid, and succinic acid.

13. A method for preparing a pharmaceutical composition of claim 1, comprising the following steps:
1) mixing vortioxetine or a pharmaceutically acceptable salt thereof and pharmaceutically acceptable excipients in a solvent, at a temperature of −50 to 150° C., to form a solution or suspension containing vortioxetine or the pharmaceutically acceptable salt and the pharmaceutical excipients, wherein the weight ratio of vortioxetine or its pharmaceutically acceptable salt to the solvent is 0.001˜100:1, and the weight ratio of vortioxetine or its pharmaceutically acceptable salt to the total pharmaceutically acceptable excipients is 1:0.1˜100; and
2) removing the solvent in the solution or suspension obtained in step 1) to obtain the composition of amorphous vortioxetine or pharmaceutically acceptable salt thereof and the pharmaceutical excipients.

14. The method of claim 13, wherein at least one of the pharmaceutical excipients is selected from the group consisting of self-excipients, propellants, solubilizers, cosolvents, emulsifiers, colorants, binders, disintegrants, fillers, lubricants, wetting agents, tonicity adjusting agents, stabilizers, glidants, flavoring agents, preservatives, suspending agents, coating materials, fragrances, anti-binders, integrators, penetration enhancers, pH adjusters, buffers, plasticizers, surfactants, foaming agents, defoamers, thickeners, humectants, absorbents, diluents, flocculants and deflocculants, antioxidants, adsorbents, filter aids, and release retardants.

15. The method of claim 13, wherein at least one of the pharmaceutical excipients is selected from the group consisting of hydroxypropylmethylcellulose, hydroxypropyl cellulose, povidone, polyethylene glycol, ethyl cellulose, liposomes, methacrylic acid copolymer, polyvinyl acetate, carboxymethylethylcellulose, carboxymethylcellulose phthalate Hydroxypropylmethylcellulose phthalate, hydroxypropylmethylcellulose acetate succinate, polyacrylic resin, carbopol, alginate, carrageenan, carboxypolactone, gums, poly pregelatinized starch, cross-linked starch, sodium carboxymethyl starch, dextrin, polyethylene oxide, chitosan, chitosan, collagen, cyclodextrin, lactose, galactose, D-mannitol, sorbitol, xylitol, urea, citric acid, tartaric acid, fumaric acid, maleic acid, and succinic acid.

16. The method of claim 13, wherein in step 1), the solvent is selected from the group consisting of alcohols having 12 or less carbon atoms, phenols, ethers, halohydrocarbons, ketones, aldehydes, nitriles, amides, sulfones, sulfoxides, carboxylic acids and water; in step 2), a method of removing the solvent includes evaporation, vacuum evaporation, spray drying, freeze-drying, hot melt extrusion, filtration, centrifugation, and agitated film drying.

17. A method for preparing a pharmaceutical composition of claim 4, comprising the following steps:
1) mixing vortioxetine hydrobromide, an organic carrier and an adsorbent in a solvent, at a temperature of −50˜150° C., to form a solution or suspension of the vortioxetine hydrobromide, organic carrier and adsorbent, wherein the weight ratio of vortioxetine hydrobromide to the solvent is from 0.001˜100:1, the weight ratio of vortioxetine hydrobromide to the organic carrier is from 1:0.1˜10, and the weight ratio of vortioxetine hydrobromide and the adsorbent is 1:0.1˜10; and
2) removing the solvent in the solution or suspension obtained in step 1) to obtain a pharmaceutical composition of amorphous vortioxetine hydrobromide salt.

18. The method of claim 17, wherein the organic carrier is selected from the group consisting of a pharmaceutically acceptable small molecule organic compound, a polymer and a copolymer.

19. The method of claim 17, wherein the organic carrier is selected from the group consisting of hydroxypropylmethylcellulose, hydroxypropylcellulose, povidone, polyethylene glycol, ethylcellulose, liposomes, methacrylic acid copolymers, polyvinyl acetate, carboxymethylethylcellulose, carboxymethylcellulose phthalate, hydroxypropyl methyl cellulose phthalate, hydroxypropylmethyl cellulose acetate succinate, polyacrylic resin, carbopol, alginate, carrageenan, carboxylactone, gum, polyvinyl alcohol, pre gelatinized starch, crosslinked starch, sodium carboxymethyl starch, dextrin, polyethylene oxide, chitosan, chitosan, collagen, cyclodextrin, lactose, galactose, D-mannitol, alcohol, xylitol, urea, citric acid, tartaric acid, fumaric acid, maleic acid and succinic acid.

20. The method of claim 17, wherein the adsorbent is selected from the group consisting of silica, aluminum oxide, titanium dioxide, magnesium oxide, calcium carbonate and zinc oxide.

21. The method claim 17, wherein the solvent in step 1) is selected from the group consisting of alcohols having 12 or less carbon atoms, phenols, ethers, halohydrocarbons, ketones, aldehydes, nitriles, amides, sulfones, sulfoxides, carboxylic acids and water; and in step 2) the method of removing the solvent includes evaporation, vacuum evaporation, spray drying, lyophilization, hot melt extrusion, filtration, centrifugation or agitation film drying.

22. A method for preparing a pharmaceutical composition of claim 5, comprising the following steps:
1) mixing vortioxetine hydrobromide salt with at least one organic carrier, at least one adsorbent and at least one pharmaceutically acceptable excipient in a solvent, at a temperature of −50 to 150° C., to form a solution or suspension, wherein the weight ratio of the vortioxetine hydrobromide salt to the solvent is 0.001˜100:1, the weight of vortioxetine hydrobromide is 20%˜80% of the total weight of solid dispersion, and the weight of the excipient is 0.1%˜80% of the total weight of the solid dispersion; and
2) removing the solvent in the solution or suspension obtained in the step 1) to obtain a pharmaceutical composition of amorphous vortioxetine hydrobromide salt.

23. A method for preparing a pharmaceutical composition of claim 5, comprising the following steps:
1) mixing vortioxetine hydrobromide salt with at least one organic carrier, at least one adsorbent, and at least one pharmaceutically acceptable excipient and solvent(s) in a fluidized bed, at a mixing temperature from 0˜150° C., to form a solution or suspension of the vortioxetine hydrobromide salt, organic carrier, adsorbent, and pharmaceutically acceptable excipient, wherein the weight ratio of vortioxetine hydrobromide salt to the solvent is 0.001˜100:1, the weight of the vortioxetine hydrobromide salt is 20%˜80% of the total weight of solid dispersion, and the weight of the pharmaceutical preparation excipient is 0.1%˜80% of the total weight of the solid dispersion; and
2) removing the solvent in the mixture obtained in step 1) to obtain a composition of amorphous vortioxetine hydrobromide salt.

24. The method of claim 22, wherein the at least one organic carrier is selected from the group consisting of pharmaceutically acceptable small organic molecules, polymers and copolymers.

25. The method of claim or 23, wherein the at least one organic carrier is selected from the group consisting of hydroxypropylmethylcellulose, hydroxypropylcellulose, povidone, polyethylene glycol, ethylcellulose, liposomes, methacrylic acid copolymers, polyvinyl acetate, carboxymethylethylcellulose, carboxymethylcellulose phthalate, hydroxyethylcellulose methyl cellulose phthalate, hydroxypropyl methylcellulose acetate succinate, polyacrylic resin, carbopol, alginate, carrageenan, carboxypolactone, gums, polyvinyl alcohol, pregelatinized starch, cross-linked starch, sodium starch glycolate, dextrin, polyethylene oxide, chitosan, and collagen.

26. The method of claim 22, wherein in step 1) the solvent is selected from the group consisting of alcohols having 12 or less carbon atoms, phenols, ethers, halogenated hydrocarbons, ketones, aldehydes, nitriles, amides, sulfones, sulfoxides, carboxylic acids and water; and in step 2) the method of removing the solvent is evaporation, evaporation in vacuo, spray drying, lyophilization, hot melt extrusion, filtration, centrifugation or agitation film drying.

27. A method of treating a mental disorder, comprising administering to subject in need thereof a therapeutically effective amount of a pharmaceutical composition of claim 1, wherein the mental disorder is selected from the group consisting of mood disorders, depression, anxiety disorders, post-traumatic stress disorder, depression with cognitive impairment, Alzheimer's disease, depression with residual symptoms, habitual pain and eating disorders.
